SEQUENCE LISTING (6 sequences)

SEQ ID NO: 1 - protein, 726 aa, Artificial Sequence
  a single fusion protein consisting of HPV proteins E6, E7 and L2.
MARHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFITTPTKLITYDNPAYEGIDVDNTLYFSSNDNSINIAPDPDFLDIVALHRPALTSRRTGIRYSRIGNKQTLRTRSGKSIGAKVHYYYDFSTIDSAEEIELQTITPSTYTTTSHAALPTSINNGLYDIYADDFITDTSTTHGAAAAAASTSLSGYIPANTTIPFGGAYNIPLVSGPDIPINITDQAPSLIPIVPGSPQYTIIADAGDFYLHPSYYMLRKRRKRLPYFFSDVSLAAHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPAAAFQDPQERPRKLPQLCTELQTTIHDGILECVYCKQQLLRREVYDFAFRDLCIVYRDGNPYAVCDKGLKFYSKISEYRHYCYSLYGTTLEQQYNKPLCDLLIRCINGQKPLCPEEKQRHLDKKQRFHNIRGRWTGRCMSCCRSSRTRRETQL

SEQ ID NO: 2 - DNA, 6999 bp, Artificial Sequence
  consisting of the coding sequences of a calreticulin (CRT) fused to the HPV E6, E7 and L2 proteins.
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcaccagaagcggactgccatgttccaggacccacaggagcgaccccggaaactgcctcagctgtgcacagaactgcagaccacaatccatgatatcattctggagtgcgtgtactgtaagcagcagctgctgcggagagaggtctatgatttcgcctttcgcgacctgtgcatcgtgtaccgagacggcaacccctatgctgtcggggataagtgcctgaaattctacagtaagatctcagaatatcggcactactgttatagcctgtacgggactaccctggagcagcagtataacaaacctctgtgcgacctgctgatcagatgtattaatggacagaagcccctgtgccctgaagagaaacagcggcacctggataagaagcagagatttcataacatcagaggcaggtggaccgggagatgtatgagcacaaggcgcgagactcagctgatgcacggcgacactcctaccctgcatgaatatatgctggacctgcagccagagacaactgatctgtacgggtatggacagctgaacgattctagtgaagaggaagatgagatcgacggacctgctggacaggcagaaccagaccgagcacactacaatattgtgacattctgctgtaagtgcgatagtactctgagactgtgcgtgcagtcaacccatgtcgatatcaggacactggaggacctgctgatgggaaccctgggcatcgtggggcccatttgctcccagaagcctaaaagagcttctgcaactcagctgtacaagacctgcaaacaggcagggacatgtcctccagacatcattcccaaggtggagggaaaaactatcgccgatcagattctgcagtatggatctatgggcgtcttctttggaggactgggaatcggaaccggaagtggaacaggaggcaggactgggtacattccactgggaacccgaccacctacagcaactgacaccctggctcctgtgcggccaccactgacagtggaccccgtcggccccagcgacccttccatcgtgtctctggtcgaggaaaccagtttcattgacgccggcgctccaacaagtgtgccctcaatccctccagatgtcagcgggttttccattaccacatctactgacactacccctgctatcctggatattaacaataccgtgacaactgtcaccacacacaacaatcccaccttcaccgacccctccgtgctgcagccacctaccccagcagagacaggaggacatttcaccctgagcagcagcactatcagcacccacaactacgaagaaatccctatggacacttaataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccat

SEQ ID NO: 3 - DNA, 6681 bp, Artificial Sequence
  consisting of the coding sequences of a signal peptide (pNGVL4a-Sig), a detox form of a HPV antigen E7, and a heat shock protein 70 (HSP70).
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcctcgacggatcttatggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcggtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagcttaagtttaaaccgctagcctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 4 - DNA, 2195 bp, Artificial Sequence
  The HPV recombinant viral vaccine, TA-HPV, is a live recombinant vaccinia viral vaccine, expressing HPV type 16 and HPV type 18 E6 and E7 oncogenes.
tttgaaaaacactatcttactcatactctagtttattatgagaaaaagtaccttataatacgtagttgggttatggtttctgagaacagatggggcacacaattcctagtgtgcccattaacaggtcttccaaagtacgaatgtctacgtgtgtgctttgtacgcacaaccgaagcgtagagtcacacttgcaacaaaaggttacaatattgtaatgggctctgtccggttctgcttgtccagctggaccatctatttcatcctcctcctctgagctgtcatttaattgcccataaccgtagagatcagttgtctctggttgcaaatctaacatatattcatgcaatgtaggtgtatctccatgcatgattcccagctgggtttctctacgtgttcttgatgatctgcaacaagacatacatcgaccggtccaccgaccccttatattatggaatctttgctttttgtccagatgtctttgcttttcttcaggacacagtggcttttgacagttaatacacctaattaacaaatcacacaacggtttgttgtattgctgttctaatgttgttccatacaaactataacaataatgtctatactcactaattttagaataaaactttaaacatttatcacatacagcatatggattcccatctctatatactatgcataaatcccgaaaagcaaagtcatatacctcacgtcgcagtaactgttgcttgcagtacacacattctaatattatatcatgtatagttgtttgcagctctgtgcataactgtggtaactttctgggtcgctcctgtgggtcctgaaacattgcagttctcttttggtccatggtggttaacaaagcttattacgatacaaacttaacggatatcgcgataatgaaataatttatgattatttctcgctttcaatttaacacaaccctcaagaacctttgtatttattttcattttttaagtatagaataaagaatctataaaaactaaaaaaattatacatcataaaccaatttcctagttgtttgtaagtcgacataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtactcaatcaatagcaatccatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttctacagtagaatcagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtaggaatattaagtatgcatggacctaaggcaacattgcaagacattgtattgcatttagagccccaaaatgaaattccggttgaccttctaggtcacgggcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaacccgggtacgtaataatctggtagatagaccatcgaatgtatttttttaactattacagaaaaaataacaaatatgg

SEQ ID NO: 5 - protein, 9 aa, Artificial Sequence
  HPV type 16 E7aa49-57 peptide
RAHYNIVTF

SEQ ID NO: 6 - protein, 9 aa, Artificial Sequence
  HPV18 E6aa67-75 peptide
KCIDFYSRI

(19) United States
(12) Patent Application Publication
(10) Pub. No.: US 2018/0250378 A1
(43) Pub. Date: Sep. 6, 2018

(54) COMBINATION OF THERAPEUTIC VACCINE AND PD-1-RELATED BLOCKADE FOR TREATING HUMAN PAPILLOMAVIRUS-ASSOCIATED DISEASES

(71) Applicant: Papivax Biotech Inc., Taipei (TW)

(72) Inventor: YUNG-NIEN CHANG, Taipei (TW)

(21) Appl. No.: 15/448,900

(22) Filed: Mar. 3, 2017

(51) Int. Cl. A61K 39/12; C07K 16/28; C07K 14/005; C12N 7/00; A61K 39/395

(52) U.S. Cl. CPC A61K 39/12; C07K 16/2818; C07K 16/2827; A61K 2039/53; C12N 7/00; A61K 39/3955; C07K 14/005

(57) ABSTRACT

The present disclosure relates to an immunostimulatory combination comprising a therapeutic vaccine and a PD-1-related blockade for treating human papillomavirus (HPV) associated diseases. The combination generates a more potent response against HPV-associate diseases by stimulating the immune response of immune cells.

### REFERENCE TO AN ELECTRONIC SEQUENCE LISTING

The contents of the electronic sequence listing (US60923-1_ST25.txt; Size: 27.8 KB; and Date of Creation: Jan. 23, 2017) is herein incorporated by reference in its entirety.

### FIELD

The present disclosure relates generally to the treatment of human papillomavirus (HPV) associated diseases. More specifically, the present disclosure relates to an immunostimulatory combination comprising a therapeutic vaccine and a PD-1-related blockade.

### BACKGROUND

HPV is a small, circular, and double-stranded DNA virus belonging to the Papillomaviridae family, having an icosahedral structure and no envelope. There are over 200 different virus types in this group. HPV types appear to be type-specific immunogens in that a neutralizing immunity to infection to one type of papillomavirus does not confer immunity against another type of HPV.

In humans, different HPV types cause distinct diseases. While most HPV infections are benign causing warts on areas of the body including the hands, feet and genitals. HPV has been indicated as the human biologic carcinogen at a higher risk of developing certain types of cancers, comprising penile, vaginal, vulva, anal, and oropharyngeal cancers.

HPV types 1, 2, 3, 4, 7, 10 and 26-29 cause benign warts in both normal and immunocompromised individuals. HPV types 5, 8, 9, 12, 14, 15, 17, 19-25, 36 and 46-50 cause flat lesions in immunocompromised individuals. HPV types 6, 11, 34, 39, 41-44 and 51-55 cause nonmalignant condylomata of the genital or respiratory mucosa. HPV types 6 and 11 are the causative agents for more than 90% of all condyloma (genital warts) and laryngeal papillomas. Other HPV types of particular interest with respect to cancer are types 31, 33, 35, 39, 45, 51, 52, 56, 58, 59, 66 and 68. HPV types 16 and 18 are those which have the highest association with cervical cancer. HPV types 31 and 45 are the types with the next highest association with a cancer risk. HPV types 16 and 18 cause epithelial dysplasia of the genital mucosa and are associated with the majority of in situ and invasive carcinomas of the cervix, vagina, vulva and anal canal.

HPV is perhaps best known for causing nearly 100% of cervical cancer cases, which remains the fourth most-deadly cancer in women worldwide. There are upwards of thirty subtypes of HPV and some of these subtypes have been associated with cervical cancer. Around 80% of cervical cancer cases are associated with HPV types 16 (˜60%) and 18 (˜20%).

The genome of HPV contains open reading frames (ORFs) called E1-E7 and L1 and L2: “E” means early, and “L” means late. L1 and L2 encode capsid proteins of HPV. The early (E) genes are associated with functions comprising virus replication and cell transformation.

The L1 protein is the major capsid protein having a molecular weight of from 55 to 60 kilo-Dalton (kDa) when measured by polyacrylamide gel electrophoresis. The L2 protein is the minor capsid protein which also has an estimated molecular weight of from 55 to 60 kDa and an apparent molecular weight of from 75 to 100 kDa. Most of the L2 protein is internal to the L1 protein. The L2 proteins are highly conserved among different papillomaviruses, especially the 10 basic amino acids at the C-terminus. The L1 ORF is highly conserved among different papillomaviruses.

HPV prophylactic vaccines have been developed and mainly used as a preventative measure against infectious diseases. Indeed, there have been several successes in the development of the prophylactic vaccines which have effectively prevented healthy, vaccinated patients by or associated with HPV infections, targeting the major capsid protein of the virus-like particles (VLPs). VLPs are morphologically similar to authentic virions and are capable of inducing high titres of neutralizing antibodies upon administration into animals or humans. Because VLPs do not contain the potentially oncogenic viral genome, they present a safe alternative to the use of live virus in HPV vaccine development. For this reason, the L1 and L2 genes have been identified as immunological targets for the development of prophylactic and therapeutic vaccines for HPV infection and disease.

After HPV viral DNA is integrated in to the host's genome, the early genes (E1, E2, E4 and, E5) and the late genes (L1 and L2) can be deleted. The E2 gene is a negative regulator for HPV oncogenes E6 and E7; therefore, these oncogenes serve as a hallmark of HPV-associated diseases because they are often expressed at elevated levels in infected cells. The oncoproteins E6 and E7 are functionally required for the initiation and maintenance of the diseases and serve as non-self, foreign proteins. For these reasons, the oncoproteins E6 and E7 have received significant attention as ideal targets for HPV therapeutic vaccines.

VLP-based vaccines have proven to be effective at inducing immune responses in human patients vaccinated with bivalent HPV types 16 and 18 prophylactic vaccines, quadrivalent HPV types 6, 11, 16, and 18 VLP-based vaccines, and nine-valent HPV types 6, 11, 16, 18, 31, 33, 45, 52, and 58 prophylactic vaccines. For example, CERVARIX, GARDASIL® and GARDASIL®9 are available polyvalent vaccines marketed for prevention of HPV.

CERVARIX is a bivalent prophylactic vaccine indicated for the prevention of HPV (types 16 and 18). CERVARIX is approved for use in females 9 through 25 years of age for the prevention of cervical cancer, cervical intraepithelial neoplasia or worse and adenocarcinoma in situ caused by HPV types 16 and 18. However, CERVARIX has not been demonstrated to provide protection against disease from vaccine and non-vaccine HPV types to which a woman has previously been exposed through sexual activity.

GARDASIL® is a commercially available quadrivalent prophylactic vaccine having activity against HPV (types 6, 11, 16, and 18). GARDASIL® is indicated in girls and women 9 through 26 years of age for the prevention of cervical, vulvar, vaginal, and anal cancer caused by HPV types 16 and 18, genital warts caused by HPV types 6 and 11, and cervical intraepithelial neoplasia, cervical adenocarcinoma in situ, cervical intraepithelial neoplasia, vulvar intraepithelial neoplasia, vaginal intraepithelial neoplasia, anal intraepithelial neoplasia caused by HPV types 6, 11, 16, and 18. GARDASIL® is also indicated in boys and men 9 through 26 years of age for the prevention of anal cancer caused by HPV types 16 and 18, genital warts caused by HPV types 6 and 11, and anal intraepithelial neoplasia caused by HPV types 6, 11, 16, and 18.

GARDASIL®9 is another commercially available nine-valent vaccine marketed for prevention of HPV (types 6, 11, 16, 18, 31, 33, 45, 52, and 58). GARDASIL®9 is indicated in girls and women 9 through 26 years of age for the prevention of cervical, vulvar, vaginal, and anal cancer caused by HPV types 16, 18, 31, 33, 45, 52, and 58, genital warts caused by HPV types 6 and 11, and cervical intraepithelial neoplasia, cervical adenocarcinoma in situ, cervical intraepithelial neoplasia, vulvar intraepithelial neoplasia, vaginal intraepithelial neoplasia, anal intraepithelial neoplasia caused by HPV types 6, 11, 16, 18, 31, 33, 45, 52, and 58. GARDASIL®9 is also indicated in boys and men 9 through 26 years of age for the prevention of anal cancer caused by HPV types 16, 18, 31, 33, 45, 52, and 58, genital warts caused by HPV types 6 and 11, and anal intraepithelial neoplasia caused by HPV types 6, 11, 16, 18, 31, 33, 45, 52, and 58.

These preventive vaccines are typically administered for systemic action, being injected into a patient subcutaneously or intramuscularly (e.g., deltoid), remote from any particular target, such as the cervix. Moreover, they are generally accepted to be effective prior to exposure to HPV and are not commonly known to be effective for treatment after exposure to, or infection with, HPV.

There are limited treatment options for patients with established HPV infections and associated diseases. In addition, the HPV infections remain extremely common globally, representing a significant health burden. Therefore, there is an urgent need to develop effective and innovative treatments to clear HPV infections and HPV-associated diseases.

HPV antigen-derived proteins are processed by dendritic cells (DCs) and presented by major histocompatibility complex (MHC) class I or class II molecules to initiate CD8+ or CD4+ T cell immune responses, respectively. Protein-based vaccines have been shown to be safe and easy to produce. However, the protein-based vaccines suffer from low immunogenicity. To overcome this setback, adjuvants and immune-stimulating molecules are often added to enhance processing and the MHC class I presentation of the protein-based vaccines.

The present disclosure relates to an immunostimulatory combination comprising a therapeutic vaccine and a PD-1-related blockade for treating HPV-associated diseases, comprising cervical cancer and head and neck cancer. The present disclosure further relates to a method of using an immunostimulatory combination comprising a therapeutic vaccine and a PD-1-related blockade for treating HPV associated diseases.

### BRIEF DESCRIPTION OF THE DRAWINGS

Implementations of the present technology will now be described, by way of example only, with reference to the attached figures. Many aspects of the present disclosure can be better understood with reference to the following figures. The components in the figures are not necessarily drawn to scale with the emphasis instead being placed upon clearly illustrating the principles of the present disclosure.

FIG. 1A shows representative flow cytometry analysis of HPV type 16 E7-specific CD8+ T cells which are activated by TA-CIN batch 0844FP pulsed with DC-1 cells, as described in EXAMPLE 1.

FIG. 1B is a summary of flow cytometry data of HPV type 16 E7-specific CD8+ T cells which are activated by TA-CIN batch 0844FP pulsed with DC-1 cells, as described in EXAMPLE 1.

FIG. 2A shows representative flow cytometry analysis of HPV type 16 E7-specific CD8+ T cells which are activated by TA-CIN batch 0907GP pulsed with DC-1 cells, as described in EXAMPLE 1.

FIG. 2B is a summary of flow cytometry data of HPV type 16 E7-specific CD8+ T cells which are activated by TA-CIN batch 0907GP pulsed with DC-1 cells, as described in EXAMPLE 1.

FIG. 3A is a schematic illustration of TA-CIN protein vaccination without adjuvant in naïve C57BL/6 mice, as described in EXAMPLE 2.

FIG. 3B shows representative flow cytometry analysis of TA-CIN protein vaccination without adjuvant in naïve C57BL/6 mice, as described in EXAMPLE 2.

FIG. 3C is a summary of flow cytometry data of TA-CIN protein vaccination without adjuvant in naïve C57BL/6 mice, as described in EXAMPLE 2.

FIG. 4A is a schematic illustration of TA-CIN protein vaccination without adjuvant in HPV type 16 E6/E7-expressing TC-1 tumor-bearing mice, as described in EXAMPLE 3.

FIG. 4B shows lungs images of TC-1 tumor-bearing mice, as described in EXAMPLE 3.

FIG. 4C is a summary of lungs weight of TC-1 tumor-bearing mice, as described in EXAMPLE 3.

FIG. 4D is a summary of the number of lungs tumor nodules from TC-1 tumor-bearing mice, as described in EXAMPLE 3.

FIG. 4E shows representative flow cytometry analysis of TA-CIN protein vaccination without adjuvant in HPV type 16 E6/E7-expressing TC-1 tumor-bearing mice, as described in EXAMPLE 3.

FIG. 4F is a summary of flow cytometry data of TA-CIN protein vaccination without adjuvant in HPV type 16 E6/E7-expressing TC-1 tumor-bearing mice, as described in EXAMPLE 3.

FIG. 5A is a schematic illustration of TA-CIN protein vaccination without adjuvant in HPV type 16 E6/E7-expressing TC-1 tumor-bearing mice in combination with PD-1 blockade, as described in EXAMPLE 4.

FIG. 5B is results of TC-1 tumor volume under each treatment regimen, as described in EXAMPLE 4.

FIG. 5C is a summary of TC-1 tumor volume, as described in EXAMPLE 4.

FIG. 5D is survival curves of TC-1 tumor-bearing mice under each treatment regimen, as described in EXAMPLE 4.

FIG. 6A is a schematic illustration of pNGVL4a-CRT/E6E7L2 DNA vaccine vaccination without adjuvant in HPV type 16 E6/E7-expressing TC-1 tumor-bearing mice in combination with PD-1 blockade, as described in EXAMPLE 5.

FIG. 6B is a summary of TC-1 tumor volume, as described in EXAMPLE 5.

FIG. 6C is survival curves of TC-1 tumor-bearing mice under each treatment regimen, as described in EXAMPLE 5.

FIG. 7A is a schematic illustration of pNGVL4a-sig/E7(detox)/HSP70 DNA vaccine vaccination without adjuvant in HPV type 16 E6/E7-expressing TC-1 tumor-bearing mice in combination with PD-1 blockade, as described in EXAMPLE 6.

FIG. 7B is a summary of TC-1 tumor volume, as described in EXAMPLE 6.

FIG. 7C is survival curves of TC-1 tumor-bearing mice under each treatment regimen, as described in EXAMPLE 6.

FIG. 8A is a schematic illustration of TA-HPV vaccinia virus vaccination with or without PD-1 blockade treatment in HPV16 E6E7-expressing TC-1 tumor-bearing mice, as described in EXAMPLE 7.

FIG. 8B shows representative flow cytometry analysis of TA-HPV vaccinia virus vaccination with or without anti-PD-1 antibody treatment in HPV16 E6E7-expressing TC-1 tumor-bearing mice, as described in EXAMPLE 7.

FIG. 8C is a summary of flow cytometry data of TA-HPV vaccinia virus vaccination with or without anti-PD-1 antibody treatment in HPV16 E6E7-expressing TC-1 tumor-bearing mice, as described in EXAMPLE 7.

FIG. 9A is a schematic illustration of TA-HPV vaccinia virus vaccination with or without PD-1 blockade treatment in HPV16 E6E7-expressing TC-1 tumor-bearing mice, as described in EXAMPLE 8.

FIG. 9B is a summary of TC-1 tumor volume, as described in EXAMPLE 8.

FIG. 9C is survival curves of TC-1 tumor-bearing mice under each treatment regimen, as described in EXAMPLE 8.

FIG. 10A is a schematic illustration of pNGVL4a-sig/E7(detox)/HSP70 DNA vaccine vaccination prime followed by TA-CIN protein vaccination boost with or without PD-1 blockade treatment in HPV16 E6E7-expressing TC-1 tumor-bearing mice, as described in EXAMPLE 9.

FIG. 10B shows representative flow cytometry analysis of pNGVL4a-sig/E7(detox)/HSP70 DNA vaccine vaccination prime followed by TA-CIN protein vaccination boost with or without PD-1 blockade treatment in HPV16 E6E7-expressing TC-1 tumor-bearing mice, as described in EXAMPLE 9.

FIG. 10C is a summary of flow cytometry data of pNGVL4a-sig/E7(detox)/HSP70 DNA vaccine vaccination prime followed by TA-CIN protein vaccination boost with or without PD-1 blockade treatment in HPV16 E6E7-expressing TC-1 tumor-bearing mice, as described in EXAMPLE 9.

FIG. 11A is a schematic illustration of pNGVL4a-sig/E7(detox)/HSP70 DNA vaccine vaccination prime followed by TA-CIN protein vaccination boost with or without PD-1 blockade treatment in HPV16 E6E7-expressing TC-1 tumor-bearing mice, as described in EXAMPLE 9.

FIG. 11B is a summary of TC-1 tumor volume, as described in EXAMPLE 9.

FIG. 11C is survival curves of TC-1 tumor-bearing mice under each treatment regimen, as described in EXAMPLE 9.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the examples described herein.

Several definitions that apply throughout this disclosure will now be presented.

The term “comprising” or “containing” when utilized, means “including, but not necessarily limited to”; it specifically indicates open-ended inclusion or membership in the so-described combination, group, series and the like. The term “administering”, “administration” and “administered” are the dispensing of a therapeutic agent to treat a condition, which is usually given orally, intravenously, parenterally, subcutaneously, intraperitoneally, intramuscularly, skin scarification, added to a “drip”, or paint on the skin or mucosal. The term “applying” is the act of bringing something into contact or of starting an action. The term “irradiated” is the process by which an object is exposed to light or radiation. The term “illumination” is the lighting up of a part, cavity, organ, or object. The term “dosage form” is pharmaceutical drug products in the form in which they are marketed for use in a particular configuration with a specific mixture of active ingredients and excipients, and apportioned into a particular dose. The term “infect” or “infection” are the invasion of bodily tissue by pathogenic microorganisms that proliferate, resulting in tissue injury that can progress to disease. The term “immunity” is the ability of an organism to resist a particular infection or toxin by the action of specific antibodies or sensitized white blood cells. The term “against” means a defense or a safeguard from pathogens infections or diseases. The term “immunocompromised” means an impaired immune system and therefore incapable of an effective immune response. The term “in situ” is means something that is in its original place. The term “in vitro” is tests or experiments are made to occur in a laboratory vessel or other controlled experimental environment rather than within a living organism or natural setting. The term “in vivo” is tests or experiments are occurring or made to occur within a living organism or natural setting. The term “open reading frame” is the part of a reading frame that has the potential to be translated in molecular genetics. An open reading frame is a continuous stretch of codons that do not contain a stop codon. The “prophylactic” is intended to prevent disease, relating to prophylaxis or prevention. The term “monoclonal” means the antibodies that are made by identical immune cells that are all clones of a unique parent cell.

“Carcinogen” means a substance capable of causing cancer in living tissue. “Oncogene” means a gene that in certain circumstances can transform a cell into a tumor cell. “Oncoprotein” means a protein encoded by an oncogene which can cause the transformation of a cell into a tumor cell if introduced into it. “Wart” is a small, hard, growth on the skin, caused by a virus infection. “Condyloma” is a raised growth on the skin resembling a wart, typically in the genital region, and transmissible by contact. “Papillomas” is a small wart-like growth on the skin or on a mucous membrane, derived from the epidermis. “Dendritic cell” is an antigen-presenting leukocyte found in the skin, mucosa, and lymphoid tissue that initiates a primary immune-response by activating lymphocytes and secreting cytokines. “Major histocompatibility complex” is a set of cell surface proteins essential for the acquired immune system to recognize foreign molecules in vertebrates, which in turn determines histocompatibility. The main function of major histocompatibility complex molecules is to bind to peptide fragments derived from pathogens and display them on the cell surface for recognition by the appropriate T-cells. “T cell” is a lymphocyte of a type produced or processed by the thymus gland and actively participating in the immune response. “Apoptosis” is a process of programmed cell death that occurs in multicellular organisms. The term “antibody” as used herein includes both polyclonal and monoclonal antibodies, as well as fragments thereof, such as, Fv, Fab and F(ab)2 fragments that are capable of binding antigen or hapten. The term “rad” is a deprecated unit of absorbed radiation dose, defined as 1 rad=0.01 Gy=0.01 J/kg. The term “pfu”, plaque-forming unit, is a measure of the number of particles capable of forming plaques per unit volume, such as virus particles. It is a functional measurement rather than a measurement of the absolute quantity of particles: viral particles that are defective or which fail to infect their target cell will not produce a plaque and thus will not be counted.

### INTRODUCTION

Papillomavirus infections occur in a variety of animals, including humans, sheep, dogs, cats, rabbits, monkeys, snakes, mice, and cows. Papillomaviruses infect epithelial cells, generally inducing benign epithelial or fibroepithelial tumors at the site of infection. Papillomaviruses may be classified into distinct groups based on the host that they infect.

Human papillomavirus (HPV) is a small, circular, and double-stranded DNA virus belonging to the Papillomaviridae family, having an icosahedral structure and no envelope. There are over 200 different virus types in this group. HPV types appear to be type-specific immunogens in that a neutralizing immunity to infection to one type of papillomavirus does not confer immunity against another type of HPV.

In humans, different HPV types cause distinct diseases. While most HPV infections are benign causing warts on areas of the body including the hands, feet and genitals. HPV has been indicated as the human biologic carcinogen at a higher risk of developing certain types of intraepithelial neoplasia or cancers, comprising penile, vaginal, vulva, anal, and oropharyngeal cancers.

HPV types 1, 2, 3, 4, 7, 10 and 26-29 cause benign warts in both normal and immunocompromised individuals. HPV types 5, 8, 9, 12, 14, 15, 17, 19-25, 36 and 46-50 cause flat lesions in immunocompromised individuals. HPV types 6, 11, 34, 39, 41-44 and 51-55 cause nonmalignant condylomata of the genital or respiratory mucosa. HPV types 6 and 11 are the causative agents for more than 90% of all condyloma (genital warts) and laryngeal papillomas. Other HPV types of particular interest with respect to cancer are types 31, 33, 35, 39, 45, 51, 52, 56, 58, 59, 66 and 68. HPV types 16 and 18 are those which have the highest association with cervical cancer. HPV types 31 and 45 are the types with the next highest association with a cancer risk. HPV types 16 and 18 cause epithelial dysplasia of the genital mucosa and are associated with the majority of in situ and invasive carcinomas of the cervix, vagina, vulva and anal canal.

HPV is perhaps best known for causing nearly 100% of cervical cancer cases, which remains the fourth most-deadly cancer in women worldwide. There are upwards of thirty subtypes of HPV and some of these subtypes have been associated with cervical cancer. Around 80% of cervical cancer cases are associated with HPV types 16 (˜60%) and 18 (˜20%).

The genome of HPV contains open reading frames (ORFs) called E1-E7 and L1 and L2: “E” means early, and “L” means late. L1 and L2 encode capsid proteins of HPV. The early (E) genes are associated with functions such as virus replication and cell transformation.

The L1 protein is the major capsid protein having a molecular weight of from 55 to 60 kilo Dalton (kDa) when measured by polyacrylamide gel electrophoresis. The L2 protein is the minor capsid protein which also has an estimated molecular weight of from 55 to 60 kDa and an apparent molecular weight of from 75 to 100 kDa. Most of the L2 protein is internal to the L1 protein. The L2 proteins are highly conserved among different papillomaviruses, especially the 10 basic amino acids at the C-terminus. The L1 ORF is highly conserved among different papillomaviruses.

HPV prophylactic vaccines have been developed and mainly used as a preventative measure against infectious diseases. Indeed, there have been several successes in the development of the prophylactic vaccines which have effectively prevented healthy, vaccinated patients by or associated with HPV infections, targeting the major capsid protein of the virus-like particles (VLPs). VLPs are morphologically similar to authentic virions and are capable of inducing high titres of neutralizing antibodies upon administration into animals or humans. Because VLPs do not contain the potentially oncogenic viral genome, they present a safe alternative to the use of live virus in HPV vaccine development. For this reason, the L1 and L2 genes have been identified as immunological targets for the development of prophylactic vaccines for HPV infection.

After HPV viral DNA is integrated in to the host's genome, the early genes (E1, E2, E4 and, E5) and the late genes (L1 and L2) can be deleted. The E2 gene is a negative regulator for HPV oncogenes E6 and E7. If E2 is destroyed when the viral genome integrates it results in expression of the viral oncogenes E6 and E7 in the host cells and cell transformation. The oncoproteins E6 and E7 are functionally required for the initiation and maintenance of the diseases. Therefore, these viral oncogenes serve as a hallmark of HPV-associated diseases because they are often expressed at elevated levels in infected cells. For these reasons, the HPV oncoproteins E6 and E7 have received significant attention as ideal targets for HPV therapeutic vaccines.

The preventive vaccines are typically administered for systemic action, being injected into a patient subcutaneously or intramuscularly (e.g., deltoid), remote from any particular target, such as the cervix. Moreover, they are generally accepted to be effective prior to exposure to HPV and are not commonly known to be effective for treatment after exposure to, or infection with, HPV.

There are limited treatment options for patients with established HPV infections and associated diseases. In addition, the HPV infections remain extremely common globally, representing a significant health burden. Therefore, there is an urgent need to develop effective and innovative treatments to clear HPV infections and HPV-associated diseases.

The present disclosure relates to an immunostimulatory combination comprising therapeutic vaccine and PD-1-related blockade for treating HPV associated diseases. The present disclosure further relates to a method of using an immunostimulatory combination comprising a therapeutic vaccine and a PD-1-related blockade for treating HPV associated diseases.

HPV antigen-derived proteins are processed by dendritic cells (DCs) and presented by major histocompatibility complex (MHC) class I or class II molecules to initiate CD8+ or CD4+ T cell immune responses, respectively. Protein-based vaccines have been shown to be safe and easy to produce. However, protein-based vaccines suffer from low immunogenicity. To overcome this setback, adjuvants and immune-stimulating molecules are often added to enhance processing and MHC class I presentation of protein-based vaccines.

A therapeutic vaccine, a PD-1-related blockade or a combination thereof may be administered by a variety of routes comprising orally, intravenously, parenterally, subcutaneously, intraperitoneally, intramuscularly, skin scarification, added to a “drip”, paint on the skin, and mucosal.

The dosage administered may vary with the condition, sex, weight, age of the individual, the route of administration, and the HPV type vaccine. The vaccine may be used in dosage form comprising capsule, suspension, elixir, and liquid solution. The capsule is an enclosing structure, usually as a solid dosage form in which a drug is enclosed in a hard or soft soluble container or “shell” of a suitable form of gelatin. The suspension is a heterogeneous mixture containing solid particles that are sufficiently large for sedimentation. The elixir is a clear, sweetened, hydro-alcoholic liquid intended for oral use, usually contains flavoring substances and is used either as vehicles or for the therapeutic effect of the active medicinal agents. The liquid solution is a homogeneous mixture composed of two or more substances (solutes) dispersed molecularly in a sufficient quantity of dissolving liquid medium (solvent).

The vaccine may be formulated with an immunologically acceptable carrier. The vaccines are administered in therapeutically effective amounts, that is, in amounts sufficient to generate an immunologically protective response. The therapeutically effective amount may vary according to the type of HPV. The vaccine may be administered in single or multiple doses.

The present disclosure makes a possible combination comprising a therapeutic vaccine and a PD-1-related blockade for preventing HPV infection and treating HPV-associated diseases. The combination, when introduced into a suitable host, is capable of inducing an immunologic response in the host.

The therapeutic vaccine aims to stimulate cell-mediated immune responses to specifically target and kill HPV-infected cells. The therapeutic vaccine targeting HPV oncogenes E6 and E7 shows particular promise in treating HPV-associated diseases. The therapeutic vaccine may comprise a TA-CIN protein, a DNA vaccine, an HPV recombinant viral vaccine, or any combination thereof in a heterologous prime-boost format.

The TA-CIN protein is a single fusion protein consisting of HPV proteins E6, E7 and L2. The TA-CIN protein proves safe and immunogenic through phase I and II clinical trials upon repeat vaccination or in prime-boost combinations with an HPV recombinant virus, TA-HPV. The TA-CIN protein induces dose-dependent HPV type 16-specific CD4, CD8 T cells, and antibodies in mice and humans. Unlike most protein vaccines, the TA-CIN protein is in filterable particulate form rather than soluble protein form; therefore, it can enter both the MHC class II pathway through exogenous antigen presentation and the MHC class I pathway through cross presentation, activating both CD4+ and CD8+ T cells, respectively. Particle size analysis shows a particle size distribution of 8-1444 nm for the TA-CIN protein. In a phase I clinical trial, the TA-CIN protein induces an HPV type 16-specific immune response in healthy volunteers with no serious adverse events reported. Protein-based vaccines are often administered with adjuvants to enhance vaccine potency; however, the TA-CIN protein is capable of inducing a strong antigen-specific CD8+ T cell response without adjuvant. Another phase I clinical trial shows that consecutive intramuscular vaccination of the TA-CIN protein in the absence of an adjuvant is safe, well-tolerated, and immunogenic in healthy patients. The TA-CIN protein may be administered in dosages ranging from 0.1 microgram per milliliter (μg/mL) to 2000 μg/mL; the TA-CIN protein may be administered preferably in dosages ranging from 1 μg/mL to 500 μg/mL; the TA-CIN protein may be administered further preferably in dosages ranging from 5 μg/mL to 200 μg/mL.

The DNA vaccine may be selected from a pNGVL4a-CRT/E6E7L2 DNA vaccine or a pNGVL4a-sig/E7(detox)/HSP70 DNA vaccine. The pNGVL4a-CRT/E6E7L2 DNA vaccine is consisting of the coding sequences of a calreticulin (CRT) fused to the HPV E6, E7 and L2 proteins. The pNGVL4a-sig/E7(detox)/HSP70 DNA vaccine is consisting of the coding sequences of a signal peptide (sig), a detox form of a HPV antigen E7, and a heat shock protein 70 (HSP70). The DNA vaccine, either the pNGVL4a-CRT/E6E7L2 DNA vaccine or the pNGVL4a-sig/E7(detox)/HSP70 DNA vaccine, may be administered in dosages ranging from 1 microgram per subject (μg/subject) to 10 milligram per subject (mg/subject); the DNA vaccine, either the pNGVL4a-CRT/E6E7L2 DNA vaccine or the pNGVL4a-sig/E7(detox)/HSP70 DNA vaccine, may be administered preferably in dosages ranging from 10 μg/subject to 5 mg/subject. The subject comprises human, mice, or other animals.

The HPV recombinant viral vaccine, TA-HPV, is a live recombinant vaccinia viral vaccine, expressing HPV type 16 and HPV type 18 E6 and E7 oncogenes. The HPV types 16 and 18 oncogenes E6 and E7 are inserted in a head-to-head orientation under the control of the p7.5 and H6 promoters at a neutral site in the vaccinia virus Wyeth strain genome. For both the HPV types 16 and 18 genes, the E6 termination codon is altered to create an E6/E7 fused open reading frame and defined mutation introduced to inactivate the Rb-binding site in E7. The recombinant viral vaccine may be administered in dosages ranging from 1×10⁴ pfu to 1×10⁷ pfu; the recombinant viral vaccine may be administered preferably in dosages ranging from 2×10⁴ pfu to 1×10⁶ pfu; the recombinant viral vaccine may be administered further preferably in dosages ranging from 1×10⁵ pfu to 5×10⁵ pfu.

Co-inhibitory molecules or immune checkpoints serve as a major mechanism through which anti-tumor immune responses and subsequent tumor progression occur. Resultantly, immune checkpoints have been widely applied to cancer immunotherapy. For example, programmed cell death protein 1 (PD-1), a cell surface receptor that belongs to the immunoglobulin superfamily and is expressed on activated T cells and pro-B cells. The PD-1 plays an important role in down regulating the immune system by preventing the activation of T-cells, which in turn reduces autoimmunity and promotes self-tolerance. The inhibitory effect of the PD-1 is accomplished through a dual mechanism of promoting apoptosis in antigen specific T-cells in lymph nodes while simultaneously reducing apoptosis in regulatory T cells. If the PD-1 binds to its ligands, PD-L1 or PD-L2, it inhibits T cell activation and function. The PD-L is usually expressed on tumor cells and immature antigen-presenting cells (APC), and is the primary ligand which the PD-1 binds to. The PD-L1 and the PD-1 interact on the surface of T cells, inhibiting T cell activation and CD8+ T cell mediated lysis. This further enables the tumor to grow and progress, which can be detrimental to the host. A PD-1 blockade (PD-1 antibody (anti-PD-1) or PD-L1 antibody (anti-PD-L1)) uses different mechanisms to elicit an antigen-specific CD8+ T cell response than therapeutic vaccines. The anti-PD-1 inhibits the activation of the PD-1 by preventing the binding of the PD-1 to ligands the PD-L1 and the PD-L2. The anti-PD-1 shows to increase anti-tumor activity in several malignancies, including melanoma, non-small cell lung cancer, bladder cancer, head and neck, renal cell carcinoma, colorectal cancer and Hodgkin's lymphoma. The PD-1 blockade, either the anti-PD-1 or the anti-PD-L1, may be administered in dosages ranging from 1 milligram per kilogram (mg/kg) to 100 mg/kg; the PD-1 blockade, either the anti-PD-1 or the anti-PD-L1, may be administered preferably in dosages ranging from 5 mg/kg to 20 mg/kg; the PD-1 blockade, either the anti-PD-1 or the anti-PD-L1, may be administered further preferably in dosage 10 mg/kg.

The present disclosure relates to the therapeutic vaccine generated more CD4+ and CD8+ T cells and induced a stronger anti-tumor effect when combined with the PD-1-related blockade. In addition, the immunotherapy with the therapeutic vaccine targeting HPV E6 and E7 combined with the PD-1 blockades can increase HPV type 16 E6/E7-specific CD8+ and CD4+ T cells responses. Furthermore, the present disclosure is the first to treat HPV type 16 E6/E7-expressing tumor using a combination comprising the therapeutic vaccine and the PD-1-related blockade.

Material and Methods

5-8 weeks old female naïve C57BL/6 mice are purchased from Charles River Laboratories (Frederick, Md.). All mice are maintained at Johns Hopkins University School of Medicine Oncology Animal Facility (Baltimore, Md.) under specific-pathogen-free conditions. All procedures are performed according to protocols approved by the Johns Hopkins Institutional Animal Care and Use Committee and in accordance with recommendations for the proper use and care of laboratory animals.

HPV type 16 E7aa49-57 peptide and HPV type 16 E6aa50-57 peptide are synthesized by GenScript (Piscataway, N.J.) at a purity of ? 80%. PE-conjugated anti-mouse CD8a (clone: 53.6.7) and FITC-conjugated anti-mouse IFN-γ (clone: XMG1.2) antibodies are purchased from BD Pharmingen (BD Pharmingen, San Diego, Calif.). Purified monoclonal anti-mouse PD-1 (clone: 29F.1A12) and monoclonal anti-mouse PD-L1 (clone: 10F.9G2) are purchased from BioXcell (West Lebanon, N.H.). Recombinant mouse IL-2 is purchased from R&D Systems (Minneapolis, Minn.). G-418 disulfate salt is purchased from Sigma-Aldrich (St. Louis, Mo.).

Description of the development of HPV type 16 E6/E7-expressing TC-1 cells (Lin K Y, Cancer Research 1996) and the TC-1 cells expressing the firefly luciferase gene (TC-1/luc) have been described previously (Huang B, Vaccine 2007). The TC-1 cell is an E6/E7-expressing murine tumor cell line. The TC-1 is tumorigenic in syngeneic, immunocompetent mice and has been characterized as a model for human cervical carcinoma. The cells are maintained in RPMI medium supplemented with 2 mM glutamine, 1 mM sodium pyruvate, 100 IU/mL penicillin, 100 μg/mL streptomycin, 400 μg/ml of G-418 and 10% fetal bovine serum (FBS). The establishment of a murine HPV type 16 E7aa49-57 peptide-specific CD8⁺ T cell line is described previously (Wang T L, et al. Gene Ther. 2000; 7:726-33). These T cells are cultured in RPMI medium supplemented with 2 mM glutamine, 1 mM sodium pyruvate, 100 IU/mL penicillin, 100 μg/mL streptomycin, 10% FBS, 0.5 ng/mL of recombinant mouse IL-2 and irradiated TC-1 cells. The T cells are re-stimulated every 7 days. DC-1 cell, an immortalized murine dendritic cell line, is described previously (Shen Z, et al. J. Immunol. 1997; 158:2723-30) and cultured in RPMI medium supplemented with 2 mM glutamine, 1 mM sodium pyruvate, 100 IU/mL penicillin, 100 μg/mL streptomycin, 10% FBS.

TA-CIN protein is a single fusion protein consisting of HPV proteins L2, E7 and E6. The TA-CIN protein is injected into mouse subcutaneously (s.c.) in 200 microliter (μL) volume at the tail base.

The DNA vaccine may be selected from a pNGVL4a-CRT/E6E7L2 DNA vaccine or a pNGVL4a-sig/E7(detox)/HSP70 DNA vaccine. The pNGVL4a-CRT/E6E7L2 DNA vaccine is consisting of the coding sequences of a calreticulin (CRT) fused to the HPV E6, E7 and L2 proteins. The pNGVL4a-sig/E7(detox)/HSP70 DNA vaccine is consisting of the coding sequences of a signal peptide (pNGVL4a-Sig), a detox form of a HPV antigen E7, and a heat shock protein 70 (HSP70).

The HPV recombinant viral vaccine, TA-HPV, is a live recombinant vaccinia viral vaccine, expressing HPV type 16 and HPV type 18 E6 and E7 oncogenes.

Table 1 shows SEQ ID NOs and the corresponding therapeutic vaccines or peptides.

TABLE 1

| SEQ ID NO: | Sequence |
|---|---|
| 1 | TA-CIN protein |
| 2 | pNGVL4a-CRT/E6E7L2 DNA vaccine |
| 3 | pNGVL4a-sig/E7(detox)/HSP70 DNA vaccine |
| 4 | Inserted sequence in TA-HPV |
| 5 | HPV type 16 E7aa49-57 peptide |
| 6 | HPV type 16 E6aa50-57 peptide |

To test the TA-CIN protein potency activating HPV16 E7-specific CD8+ T cells in vitro, a standard operating procedure is established. DC-1 cells (1×10⁶ cells in 1 mL volume per well of 6-well plate) are pulsed with the TA-CIN protein or the E7aa49-57 peptide at indicated concentration, and incubated at 37° C. for 20 hours. The DC-1 cells are then harvested and irradiated with a γ irradiator (10000 rads). After washing, 3×10⁵ of E7aa49-57 peptide-specific CD8+ T cells (day 6 after the stimulation) are co-cultured with 3×10⁵ of the above irradiated DC-1 cells at the presence of GolgiPlug (1 μL/mL) (BD Pharmingen, San Diego, Calif.) in a well of 96-well round-bottom plate in 200 μL volume at 37° C. for 20 hours. The GolgiPlug is a protein transport inhibitor containing brefeldin A, to in vitro- or in vivo-stimulated lymphoid cells blocks their intracellular protein transport processes. This results in the accumulation of cytokines and/or proteins in the Golgi complex. This increased accumulation of cytokines in the cell enhances the detectability of cytokine-producing cells with immunofluorescent staining and flow cytometric analysis. The HPV16 E7aa49-57 peptide (1 μg/mL) was directly added to the E7aa49-57 peptide-specific CD8+ T cells used as positive control. Each sample is tested triplicate. The cells of the each sample are harvested and supernatant is removed after centrifugation. The cells of the each sample are stained with PE-conjugated anti-mouse CD8a (clone 53.6.7, 1 μL per sample in the total volume of 20 μL) at 4° C. for 30 minutes. The cells of the each sample are washed with 2 mL of PBS containing 0.5% BSA and 5 mM of EDTA. Subsequently, 250 μL of Cytofix/Cytoperm (BD Pharmingen, San Diego, Calif.) are added into the each sample and incubated at 4° C. for 30 minutes. The cells of the each sample are washed with 2 mL of Cytofix/Cytoperm wash buffer. The cells of the each sample are then stained with FITC-conjugated anti-mouse IFN-γ (clone XMG1.2, 1 μL/sample) at 4° C. for 45 minutes. Subsequently, the cells of the each sample are washed with 2 mL of Cytofix/Cytoperm wash buffer and then suspended in 300 μL per sample of PBS containing 0.5% BSA and 5 mM of EDTA. The cells of the each sample are acquired with FACSCalibur flow cytometer and analyzed with CellQuest Pro software (BD biosciences, Mountain View, Calif.).

To detect the HPV16 E7-specific CD8+ T cell responses in vivo by IFN-γ intracellular staining assay and flow cytometry analysis, splenocytes from the naïve C57BL/6 mice are stimulated with the HPV16 E7aa49-57 peptide (1 μg/mL) at the presence of GolgiPlug (1 μL/mL) at 37° C. overnight. The IFN-γ intracellular staining assay and flow cytometry analysis is then performed as described above. The HPV16 E7-specific CD8+ T cells are represented as CD8+ IFN-γ+ cells.

To evaluate the potency of tumor treatment of the TA-CIN protein in a hematologic spread model, the 5-8 weeks old female naïve C57BL/6 mice are injected with 5×10⁴ of the TC-1 cells intravenously via the tail vein. Three days later, mice are vaccinated with the TA-CIN protein via subcutaneous injection and boosted twice with the same regimen at one-week intervals. Twenty-one days later, mice are sacrificed, splenocytes of the mice are prepared to detect HPV16 E6- and E7-specific CD8+ T cell responses, and lungs are harvested to examine the tumor growth.

To detect TC-1 tumor treatment, 1×10⁵ of the TC-1 cells are injected subcutaneously into the naïve C57BL/6 mice. On day 5, the TC-1 tumor-bearing mice are vaccinated with the TA-CIN protein vaccine (25 μg/200 μL per mouse) subcutaneously and boosted once 6 days later. For the PD-1-related blockade experiment, the TC-1 tumor-bearing mice are injected on day 5 with 10 mg/kg of either anti-PD-1 or anti-PD-L1 as indicated intraperitoneally (IP) and repeated every 3 days. Tumor growth is monitored by visual inspection and measurement of tumor diameter with calipers twice each week. Tumor volume is calculated using the formula [largest diameter×(perpendicular diameter)2]×π/6. Tumor survival is recorded as either natural death or a tumor diameter greater than 2 cm.

To detect TC-1 tumor treatment, 1×10⁵ of the TC-1 cells are injected subcutaneously into the naïve C57BL/6 mice. On day 3, two groups of the mice are treated with 10 mg/kg of anti-PD-1 (clone 29F.1A12) via intraperitoneally injection and repeated every two days. On day 7, one group of anti PD-1 treated and one group of anti PD-1 untreated mice are vaccinated with 50 μg/mouse of a pNGVL4a-CRT/E6E7L2 DNA vaccine via intramuscular injection or a pNGVL4a-sig/E7(detox)/HSP70 DNA vaccine via intramuscular injection. Tumor growth is monitored by measuring diameters with a digital caliper twice a week and tumor volume is calculated.

All data are expressed as means±standard deviations (SD). Comparisons between individual data point and data for tumor treatment experiments are evaluated by analysis of variance (ANOVA). Survival distributions for mice in different groups are compared by the Kaplan-Meier curves and by use of the long-rank tests. A p-value of less than 0.05 is considered significant.

### Example 1

The DC-1 cells are pulsed with the TA-CIN protein batch 0844FP or batch 0907GP at concentrations of 6.25, 12.5, 25.0, 50.0, or 100 μg/mL, and incubate at 37° C. for 20 hours. The DC-1 cells are pulsed with the E7aa49-57 peptide at concentration of 2 μg/mL, and incubate at 37° C. for 20 hours. The DC-1 cells are then harvested, irradiated (10000 rads), and washed.

The 3×10⁵ of E7aa49-57 peptide-specific CD8+ T cells (day 6 after the stimulation) are co-cultured with 3×10⁵ of the above irradiated DC-1 cells at the presence of GolgiPlug (1 μL/mL) in a well of 96-well round-bottom plate in 200 μL volume at 37° C. for 20 hours. The E7aa49-57 peptide (1 μg/mL) is directly added to the E7aa49-57 peptide-specific CD8+ T cells as a positive control. The cells are then harvested. The supernatant is removed after centrifugation.

The cells of the each sample are stained with the PE-conjugated anti-mouse CD8a (1 μL/sample in the total volume of 20 μl) at 4° C. for 30 minutes. The cells of the each sample are washed with 2 mL of the PBS containing 0.5% BSA and 5 mM of EDTA. Subsequently, 250 μL of the Cytofix/Cytoperm are added into the each sample and the cells of the each sample are incubated at 4° C. for 30 minutes. The cells of the each sample are washed with 2 mL of the Cytofix/Cytoperm wash buffer. The cells of the each sample are then stained with the FITC-conjugated anti-mouse IFN-γ (1 μL/sample) at 4° C. for 45 minutes. Subsequently, the cells of the each sample are washed with 2 mL of the Cytofix/Cytoperm wash buffer and then suspended in 300 μL/sample of the PBS containing 0.5% BSA and 5 mM of EDTA. The cells of the each sample are acquired with the FACSCalibur flow cytometer and analyzed with the CellQuest Pro software.

The flow cytometry is performed on the HPV16 E7-specific CD8+ T cells which are activated by the TA-CIN protein batch 0844FP or batch 0907GP pulsed with the DC-1 cells.

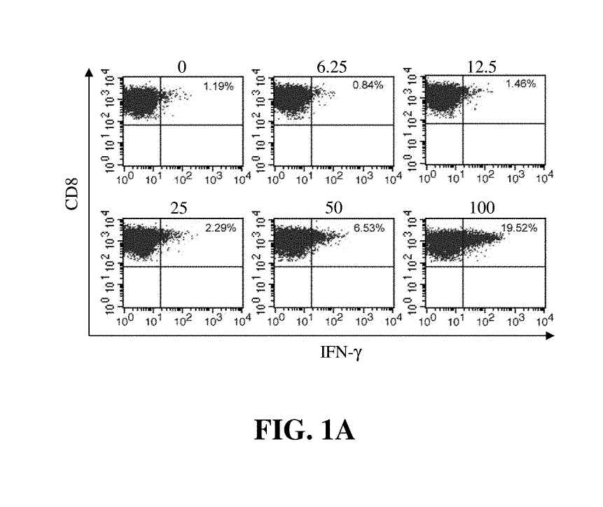

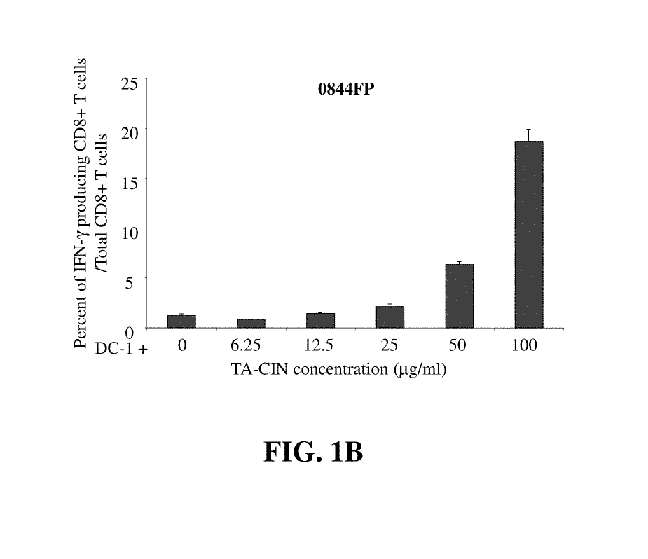

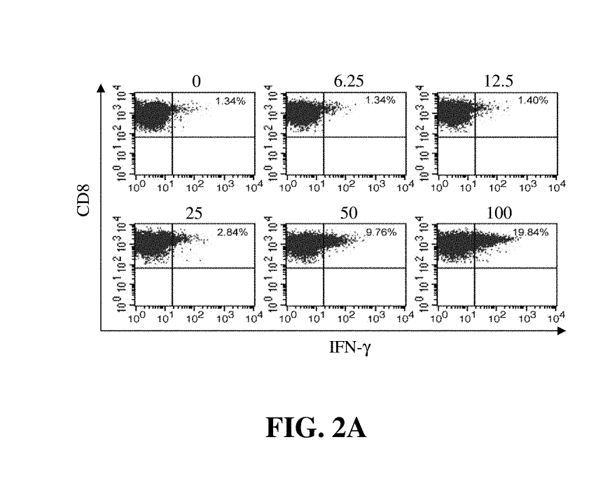

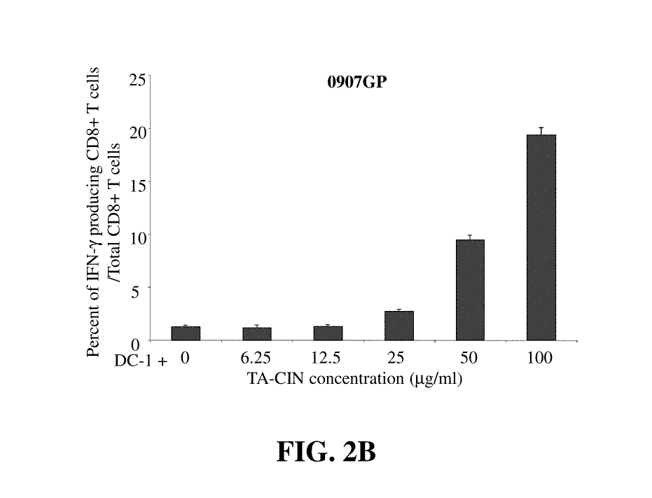

FIGS. 1A and 1B illustrate that the DC-1 cells pulsed with the TA-CIN protein batch 0844FP are able to elicit the HPV type 16 E7-specific CD8+ T cell response. FIG. 1A shows the representative the flow cytometry images of the HPV type 16 E7-specific CD8+ T cells which are activated by the DC-1 cells pulsed with the TA-CIN protein batch 0844FP. FIG. 1B shows a quantitative summary of the flow cytometry data of the HPV type 16 E7-specific CD8+ T cells which are activated by the DC-1 cells pulsed with the TA-CIN protein batch 0844FP. FIGS. 2A and 2B illustrate that the DC-1 cells pulsed with the TA-CIN batch 0907GP are also able to stimulate the HPV type 16 E7-specific CD8+ T cell response. FIG. 2A shows the representative the flow cytometry images of the HPV type 16 E7-specific CD8+ T cells which are activated by the DC-1 cells pulsed with the TA-CIN protein batch 0907GP. FIG. 2B shows a quantitative summary of the flow cytometry data of the HPV16 E7-specific CD8+ T cells which are activated by the DC-1 cells pulsed with the TA-CIN protein batch 0907GP.

The TA-CIN protein batch 0844FP and batch 0907GP show very similar response rates of the HPV type 16 E7-specific CD8+ T cell. Furthermore, the DC-1 cells pulsed with higher concentrations of the TA-CIN protein (50 or 100 μg/mL) show greater amounts of the activated HPV type 16 E7-specific CD8+ T cells. These results suggest the DC-1 cells pulsed with the TA-CIN protein are able to process and present E7 antigens through the MHC class I pathway to activate the HPV type 16 E7-specific CD8+ T cells.

### Example 2

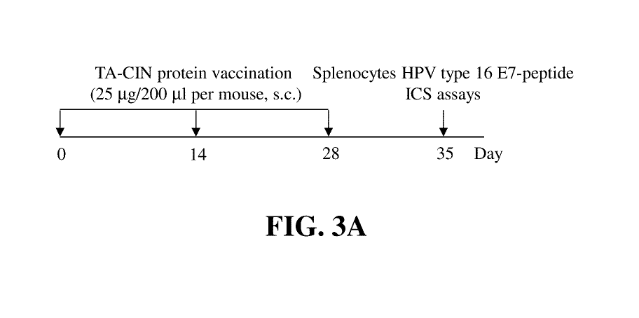

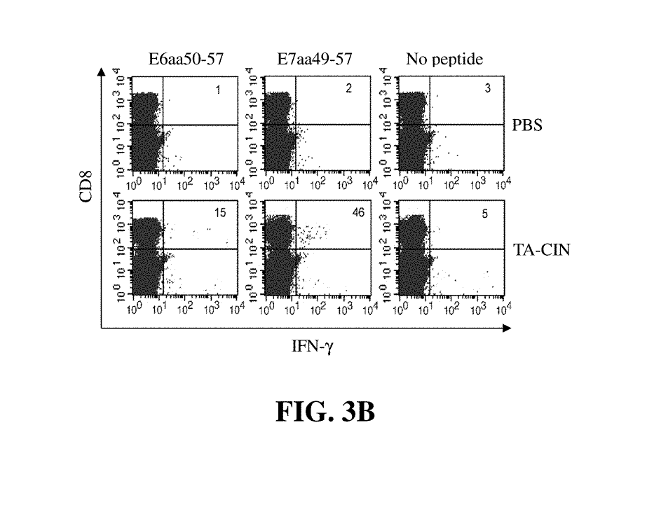

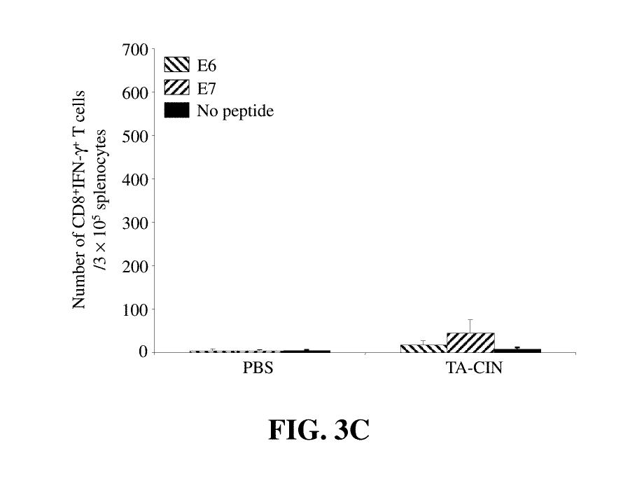

FIG. 3A illustrates the schematic of the TA-CIN protein vaccination without adjuvant in the naïve C57BL/6 mice. The 5-8 week old female naïve C57BL/6 mice (5 mice per group) are vaccinated subcutaneously with 25 μg/200 μL of the TA-CIN protein or the volume PBS. The mice are further boosted with the same regimen on day 14 and day 28. Seven days after the last vaccination booster, the splenocytes are prepared from the mice, and the splenocytes are stimulated with the either 1 μg/mL of the E6aa50-57 or the E7aa49-57 peptide in the presence of GolgiPlug (1 μL/mL) overnight. The splenocytes are then stained with the PE-conjugated anti-mouse and the FITC-conjugated anti-mouse IFN-γ. FIGS. 3B and 3C illustrate that the flow cytometry is performed on the splenocytes with the E6aa50-57 peptide, the E7aa49-57 peptide, or without peptide. FIG. 3B shows the representative flow cytometry images of the TA-CIN protein vaccination without adjuvant in the naïve C57BL/6 mice generated weak CD8+ T cell responses. FIG. 3C shows a quantitative summary of flow cytometry data of the TA-CIN protein vaccination without adjuvant in the naïve C57BL/6 mice generated weak CD8+ T cell responses.

The TA-CIN protein vaccination in the absence of an adjuvant failed to generate a significant amount of antigen-specific T cells. While some HPV type 16 E7-specific CD8+ T cells are generated, these results show that the vaccination of the naïve mice with the TA-CIN protein without an adjuvant generated a weak CD8+ T cell response in mice.

### Example 3

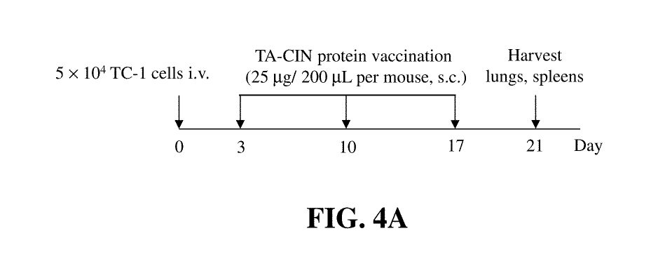

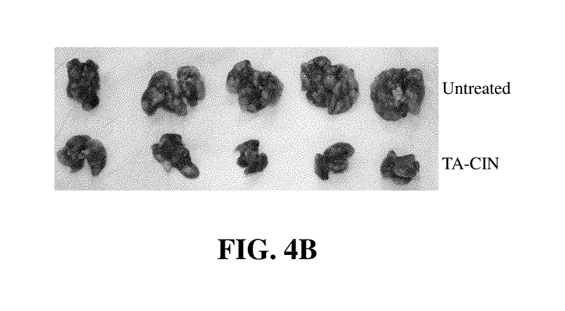

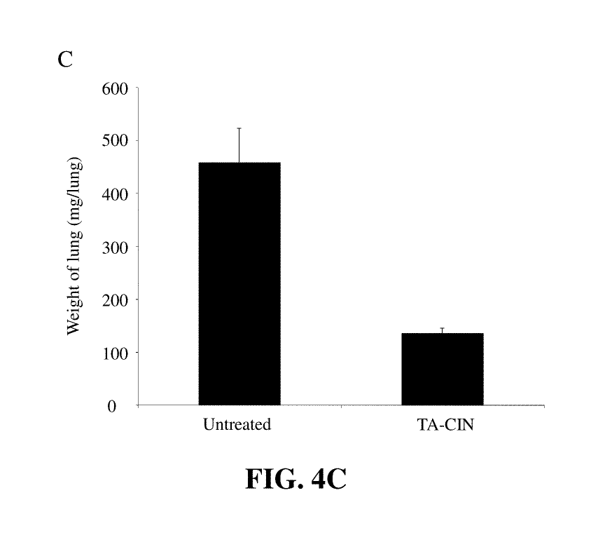

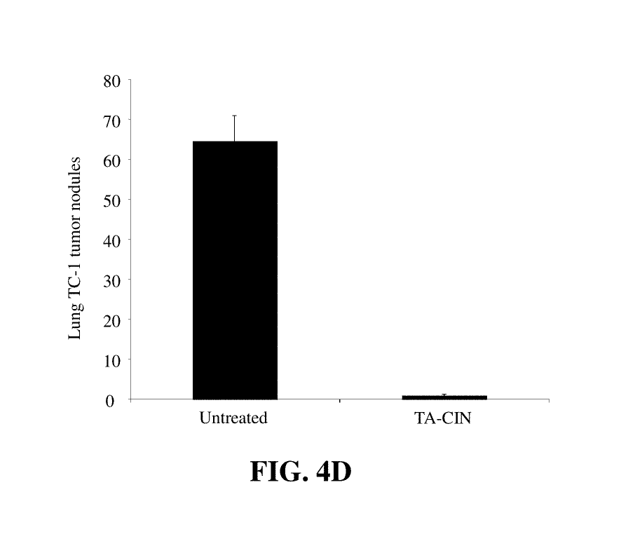

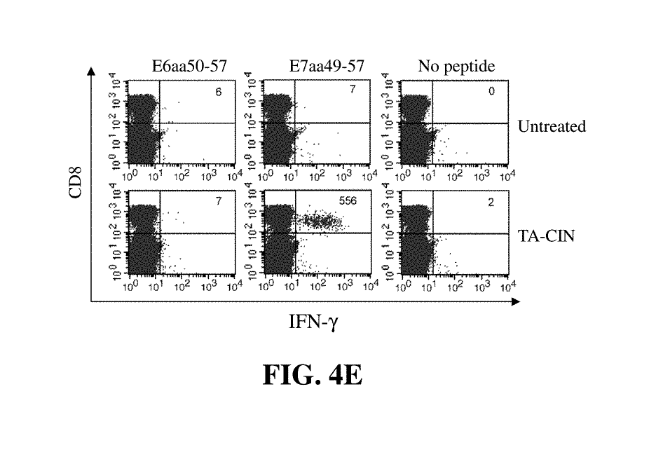

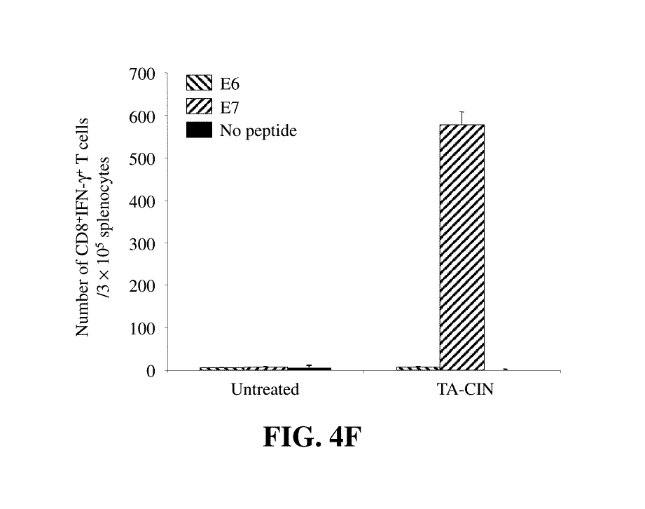

FIG. 4A illustrates the schematic of the TA-CIN protein vaccination without adjuvant in the HPV16 E6/E7-expressing TC-1 tumor-bearing mice The 5-8 week old female naïve C57BL/6 mice (5 mice per group) are injected intravenously with the TC-1 cells on day 0. On day 3, the mice are vaccinated subcutaneously with 25 g/200 μL of the TA-CIN protein or the same volume PBS. The mice then receive a vaccination booster at day 10 and day 17 using the same regimen. On day 21, the mice are sacrificed. The lungs and the spleens of the mice are harvested. The lungs weight of both vaccinated and untreated mice is measured, respectively. The tumor nodules number on the lungs of both vaccinated and untreated mice is counted and measured, respectively. FIG. 4B shows the lungs images from the TC-1 tumor-bearing mice. FIG. 4C illustrates a quantitative summary of the lungs weight of the TC-1 tumor-bearing mice comprising vaccinated and untreated mice. FIG. 4D illustrates a quantitative summary of the tumor nodules number on the lungs from the TC-1 tumor-bearing mice comprising vaccinated and untreated mice.

The splenocytes of the spleens are stimulated with either 1 μg/mL of the E6aa50-57, or the E7aa49-57 peptide at the presence of GolgiPlug (1 μL/mL) overnight. The splenocytes are then stained with the PE-conjugated anti-mouse and the FITC-conjugated anti-mouse IFN-γ. The vaccine-specific T cell responses are analyzed with the flow cytometry. FIG. 4E shows the representative flow cytometry analysis of the TA-CIN protein vaccination without adjuvant in the HPV type 16 E6/E7-expressing the TC-1 tumor-bearing mice. FIG. 4F illustrates a quantitative summary of the flow cytometry data of the TA-CIN protein vaccination without adjuvant in the HPV type 16 E6/E7-expressing TC-1 tumor-bearing mice.

The TA-CIN protein vaccination without adjuvant in the HPV type 16 E6E7-expressing TC-1 tumor-bearing mice is shown to control tumor growth. The TA-CIN protein vaccinated mice have smaller lung volumes and fewer tumor nodules than untreated mice (FIGS. 4B-4D). The TA-CIN protein treatment is able to generate a robust response of the HPV type 16 E7-specific CD8+ T cell in the TC-1 tumor-bearing mice (FIG. 4F).

### Example 4

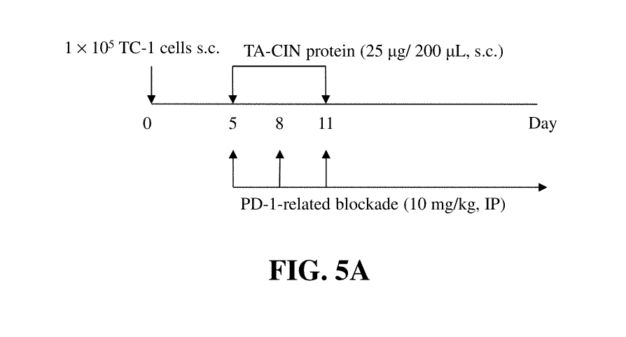

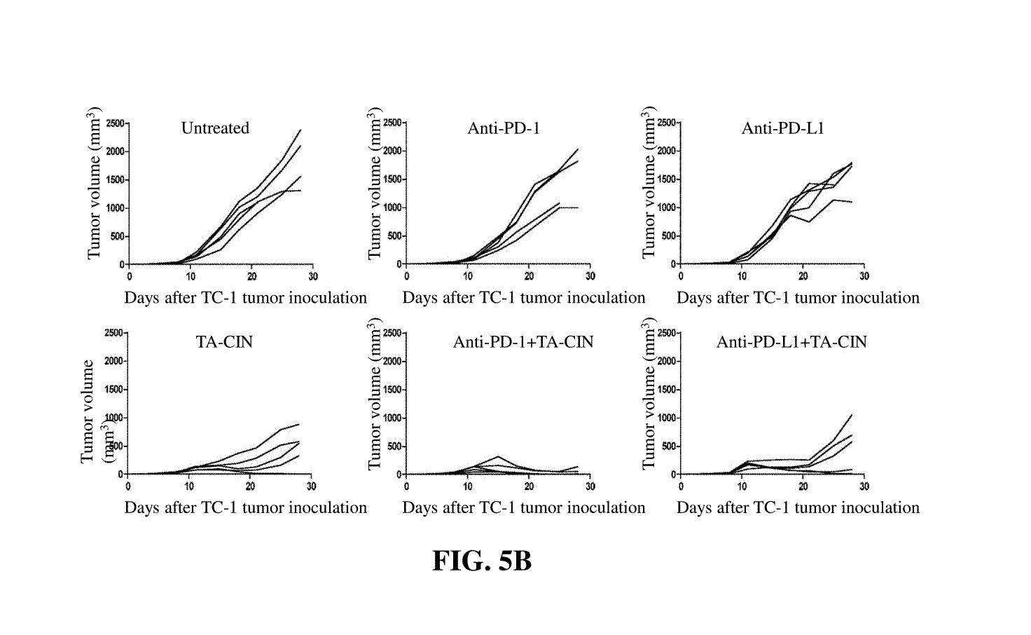

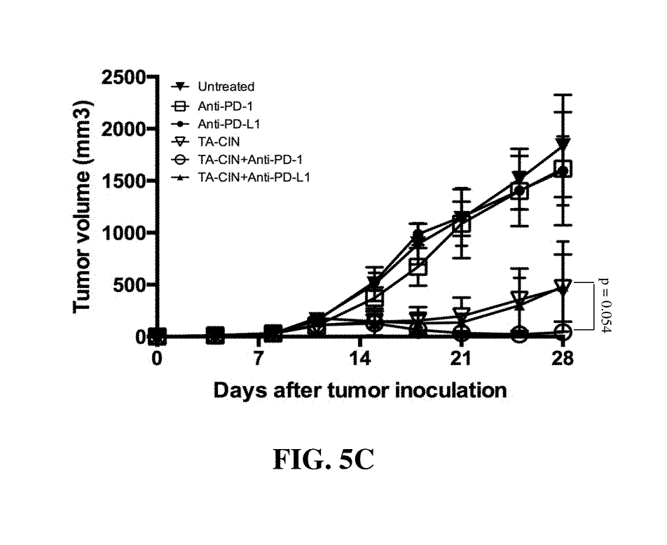

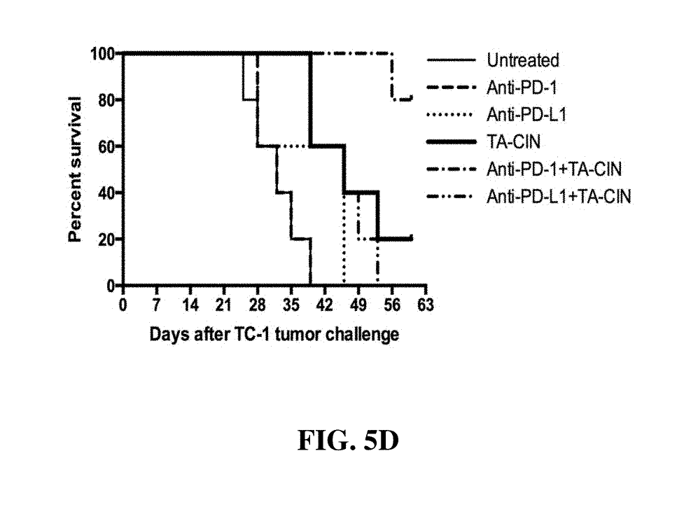

The effects of anti-PD-1 and anti-PD-L1 on tumor volume in the TA-CIN protein vaccinated and the untreated TC-1 tumor-bearing mice are tested. FIG. 5A illustrates a schematic of the TA-CIN protein vaccination without adjuvant in the HPV type 16 E6/E7-expressing TC-1 tumor-bearing mice in combination with the PD-1 blockade. The 5-8 week old female naïve C57BL/6 mice (5 mice per group) are injected subcutaneously with 1×10⁵ TC-1 cells on day 0. On day 5, mice are vaccinated with either 25 μg/200 μL per mouse of the TA-CIN protein subcutaneously, 10 mg/kg of anti-PD-1 intraperitoneally, 10 mg/kg of anti-PD-L1 intraperitoneally, or both the TA-CIN protein with the anti-PD-1 or anti-PD-L1. Administration of the anti-PD-1 or the anti-PD-L1 is repeated every three days. Mice vaccinated with the TA-CIN protein are boosted on day 11. Untreated mice are the blank control. FIG. 5B shows results of TC-1 tumor volume under different treatment regimen including the untreated, treated the anti-PD-1 without the TA-CIN protein vaccination, treated the anti-PD-L1 without the TA-CIN protein vaccination, treated the TA-CIN protein without the anti-PD-1 or the anti-PD-L1, treated the anti-PD-1 with the TA-CIN protein vaccination, and treated the anti-PD-L1 with the TA-CIN protein vaccination. FIG. 5C illustrates a quantitative summary of the TC-1 tumor volume in the HPV type 16 E6/E7-expressing TC-1 tumor-bearing mice. The tumor growth is measured and recorded using a digital caliper twice a week. FIG. 5D summaries the duration of survival of the HPV type 16 E6/E7-expressing TC-1 tumor-bearing mice receiving each treatment regimens.

The mice administered with anti-PD-1 or anti-PD-L1 alone have comparable tumor volumes on day 28 to the untreated mice. The mice received the TA-CIN protein vaccination alone and the TA-CIN protein in conjunction with the anti-PD-L1 show regression in the tumor volume. The mice vaccinated with the TA-CIN protein in conjunction with the anti-PD-1 show the lowest tumor volumes. Furthermore, the mice vaccinated with the TA-CIN protein in conjunction with the anti-PD-1 show the longest overall survival. These results suggest that vaccination with the TA-CIN protein in conjunction with the anti-PD-1 is the most effective at controlling tumor growth in the HPV type 16 E6/E7-expressing TC-1 tumor-bearing mice (FIGS. 5B-5D).

### Example 5

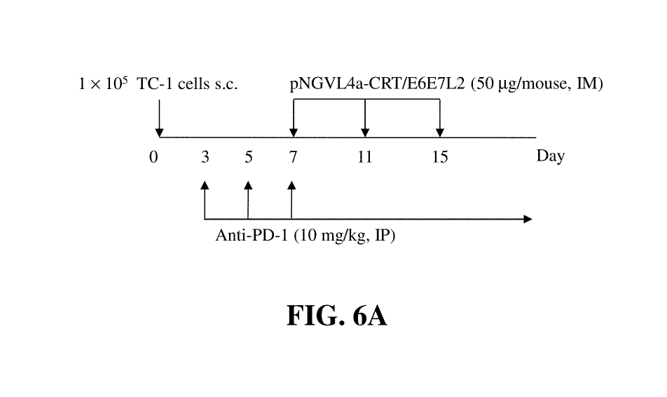

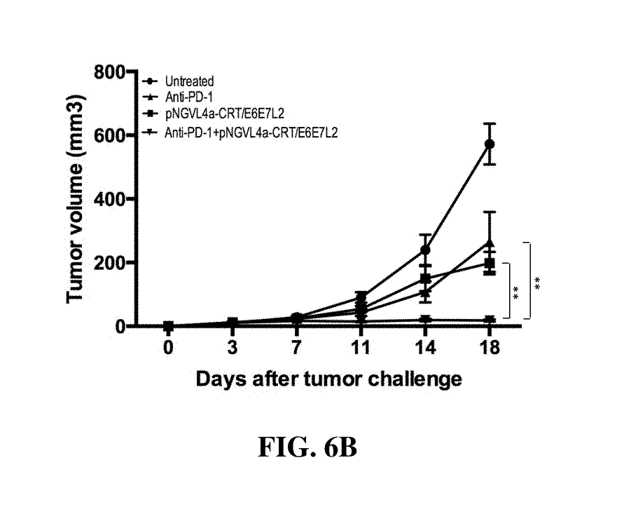

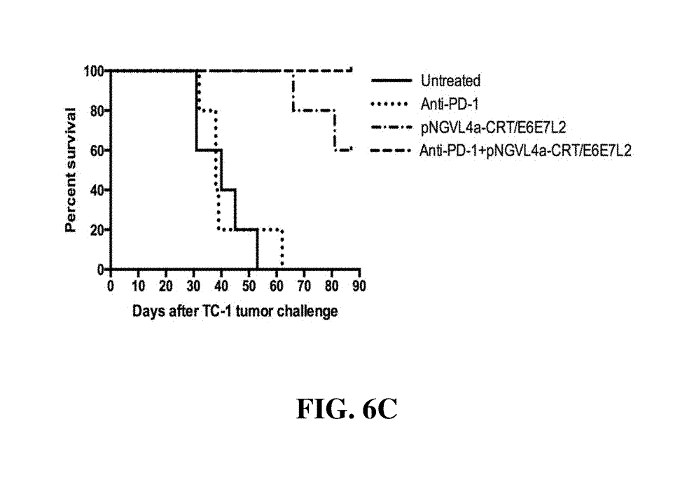

FIG. 6A illustrates a combination vaccination of a PD-1 blockade and a DNA vaccine in HPV16 E6/E7-expressing TC-1 tumor-bearing mice. The 5-8 week old female naïve C57BL/6 mice (5 mice per group) are injected with 1×10⁵ TC-1 cells subcutaneously on day 0. On day 3, two groups of the mice are treated with 10 mg/kg of anti-PD-1 via intraperitoneally (IP) injection and repeated every two days. On day 7, one anti-PD-1 treated group mice and one anti-PD-1 untreated group mice are vaccinated with 50 μg/mouse of pNGVL4a-CRT/E6E7L2 DNA vaccine via intramuscular (IM) injection. Administration of the pNGVL4a-CRT/E6E7L2 DNA vaccine is repeated every four days. FIG. 6B illustrates a quantitative summary of the TC-1 tumor volume in HPV type 16 E6/E7-expressing TC-1 tumor-bearing mice. Tumor growth is monitored by measuring diameters with a digital caliper twice a week and tumor volume is calculated. FIG. 6C summaries the duration of survival of the HPV type 16 E6/E7-expressing TC-1 tumor-bearing mice receiving each treatment regimens.

The mice administered with anti-PD-1 alone have comparable tumor volumes on day 18 to the pNGVL4a-CRT/E6E7L2 DNA vaccine treated mice. The mice received with the pNGVL4a-CRT/E6E7L2 DNA vaccine in conjunction with the anti-PD-1 show the lowest tumor volumes. Furthermore, the mice vaccinated with the pNGVL4a-CRT/E6E7L2 DNA vaccine in conjunction with the anti-PD-1 show the longest overall survival. These results suggest that vaccination with the pNGVL4a-CRT/E6E7L2 DNA vaccine in conjunction with the anti-PD-1 is the most effective at controlling tumor growth in the HPV type 16 E6/E7-expressing TC-1 tumor-bearing mice (FIGS. 6B-6C).

### Example 6

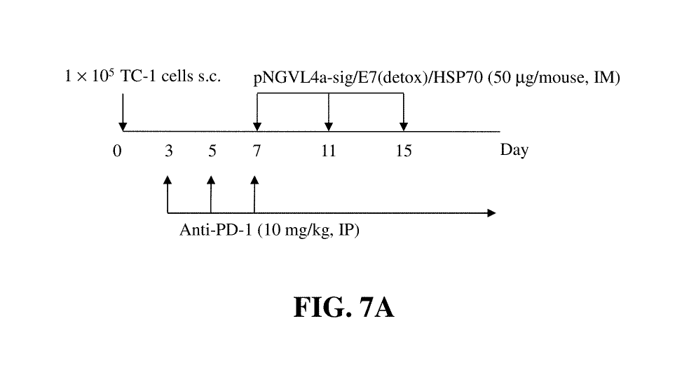

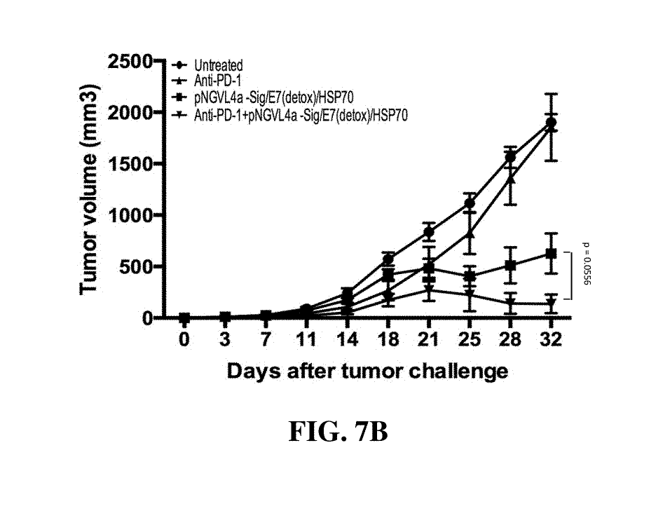

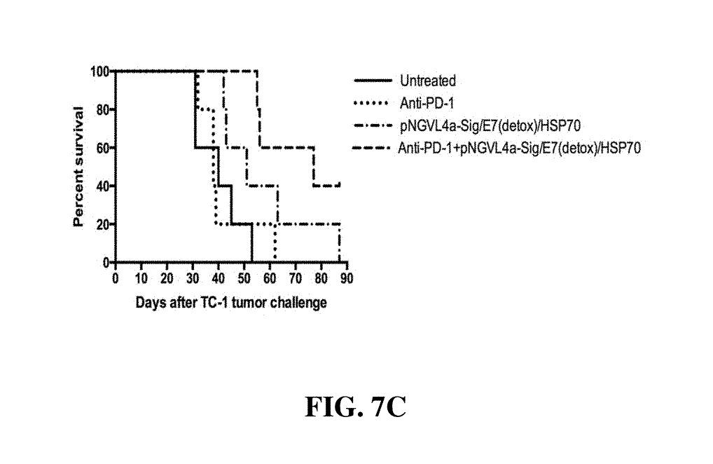

FIG. 7A illustrates a combination vaccination of a PD-1 blockade and a DNA vaccine in HPV type 16 E6/E7-expressing TC-1 tumor-bearing mice. The 5-8 week old female naïve C57BL/6 mice (5 mice per group) are injected with 1×10⁵ TC-1 cells subcutaneously on day 0. On day 3, two groups of the mice are treated with 10 mg/kg of anti-PD-1 via intraperitoneally (IP) injection and repeated every two days. On day 7, one anti-PD-1 treated group mice and one anti-PD-1 untreated group mice are vaccinated with 50 μg/mouse of pNGVL4a-sig/E7(detox)/HSP70 DNA vaccine via intramuscular (IM) injection. Administration of the pNGVL4a-sig/E7(detox)/HSP70 DNA vaccine is repeated every four days. FIG. 7B illustrates a quantitative summary of the TC-1 tumor volume in HPV type 16 E6/E7-expressing TC-1 tumor-bearing mice. Tumor growth is monitored by measuring diameters with a digital caliper twice a week and tumor volume is calculated. FIG. 7C summaries the duration of survival of HPV type 16 E6/E7-expressing TC-1 tumor-bearing mice receiving each treatment regimens.

The mice treated with the pNGVL4a-sig/E7(detox)/HSP70 DNA vaccine in conjunction with the anti-PD-1 show the lowest tumor volumes. Furthermore, the mice vaccinated with the pNGVL4a-sig/E7(detox)/HSP70 DNA vaccine in conjunction with the anti-PD-1 show the longest overall survival. The results suggest that vaccination with the pNGVL4a-sig/E7(detox)/HSP70 DNA vaccine in conjunction with the anti-PD-1 provides the most effective anti-tumor immunity and control of tumor growth in the HPV type 16 E6/E7-expressing TC-1 tumor-bearing mice (FIGS. 7B-7C).

### Example 7

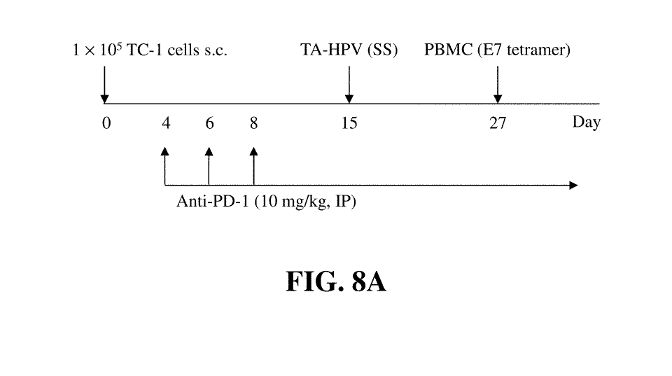

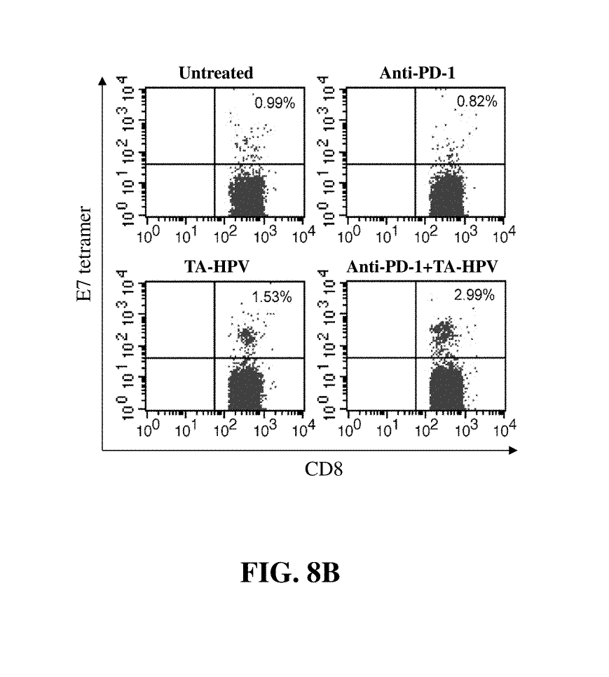

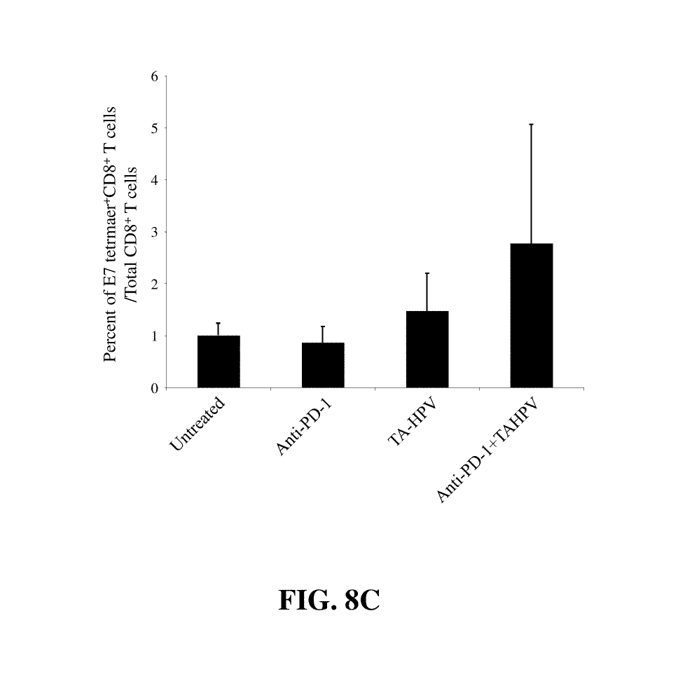

FIG. 8A illustrates of a combination vaccination of a PD-1 blockade and the TA-HPV vaccinia virus in HPV type 16 E6/E7-expressing TC-1 tumor-bearing mice. The 5-8 week old female naïve C57BL/6 mice (5 mice per group) are injected with 1×10⁵ TC-1 cells subcutaneously on day 0. On day 4, two groups of the tumor-bearing mice are injected with 10 mg/kg of the anti-PD-1 via intraperitoneally (IP) and repeated every two days. On day 15 after tumor challenge, one group of untreated and one group of the anti-PD-1 treated mice are vaccinated with 1×10⁵ pfu of the TA-HPV vaccinia virus on the tail skin (1 cm from the tail base) through scarification (SS). 12 days after the TA-HPV vaccinia virus vaccination, peripheral blood mononuclear cells (PBMCs) are prepared from the mice, and stained with purified anti-mouse CD16/32 antibody first. The PBMCs are then stained with FITC-conjugated anti-mouse CD8a antibody and PE-conjugated HPV16 E7aa49-57 peptide-loaded H-2D^{b} tetramer. The cells are acquired with FACSCalibur and analyzed with CellQuest Pro software. FIG. 8B illustrates a representative flow cytometry analysis of the TA-HPV vaccinia virus vaccination with or without anti-PD-1 treatment in the HPV type 16 E6/E7-expressing the TC-1 tumor-bearing mice. FIG. 8C illustrates a quantitative summary of the flow cytometry data of the TA-HPV vaccinia virus vaccination with or without anti-PD-1 treatment in the HPV type 16 E6/E7-expressing TC-1 tumor-bearing mice. The TA-HPV vaccinia virus in conjunction with the anti-PD-1 treatment is able to generate a robust response of the HPV type 16 E7-specific CD8+ T cell in the TC-1 tumor-bearing mice.

### Example 8

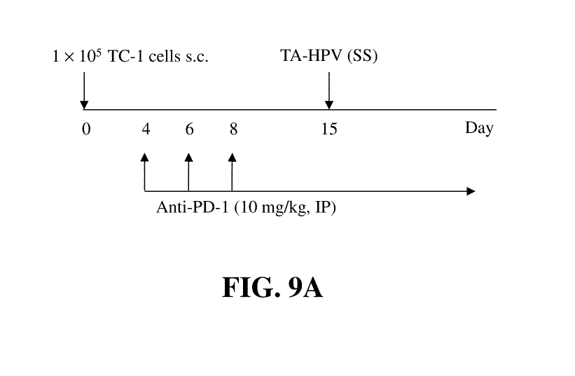

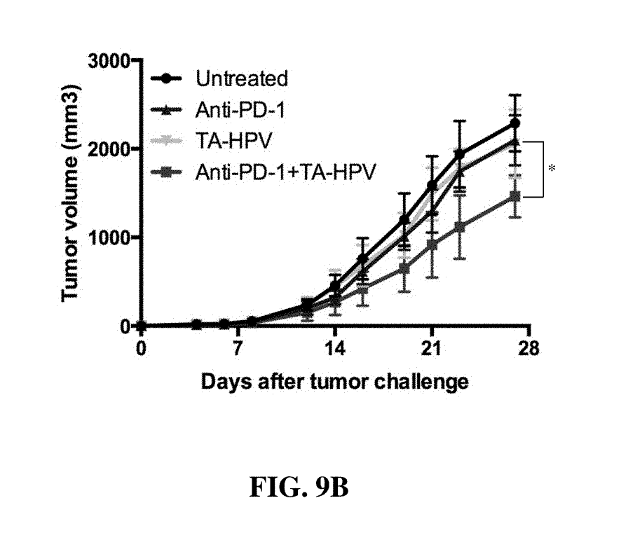

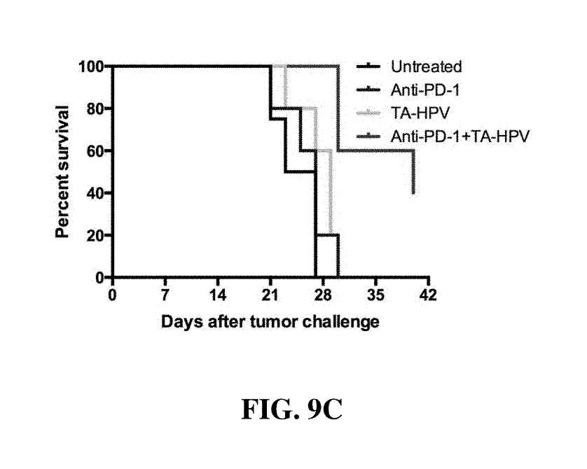

FIG. 9A illustrates a combination vaccination of a PD-1 blockade and the TA-HPV vaccinia virus in HPV type 16 E6/E7-expressing TC-1 tumor-bearing mice. The 5-8 week old female naïve C57BL/6 mice (5 mice per group) are injected with 1×10⁵ TC-1 cells subcutaneously on day 0. On day 4, two groups of the tumor-bearing mice are injected with 10 mg/kg of the anti-PD-1 via intraperitoneally (IP) and repeated every two days. On day 15 after tumor challenge, one group of untreated and one group of the anti-PD-1 treated mice are vaccinated with 1×10⁵ pfu of the TA-HPV vaccinia virus on the tail skin (1 cm from the tail base) through scarification (SS). The tumor growth is monitored twice a week with a digital caliper. FIG. 9B illustrates a quantitative summary of the TC-1 tumor volume in HPV type 16 E6/E7-expressing TC-1 tumor-bearing mice. The death of the mouse is recorded as either natural death or when the tumor size over 2 cm in diameter. FIG. 9C illustrates the duration of survival of HPV type 16 E6/E7-expressing TC-1 tumor-bearing mice receiving each treatment regimens.

The mice treated with the TA-HPV vaccinia virus in conjunction with the anti-PD-1 show the lowest tumor volumes. Furthermore, the mice vaccinated with the TA-HPV vaccinia virus in conjunction with the anti-PD-1 show the longest overall survival. The results suggest that vaccination with the TA-HPV vaccinia virus in conjunction with the anti-PD-1 provides the most effective anti-tumor immunity and control of tumor growth in the HPV type 16 E6/E7-expressing TC-1 tumor-bearing mice (FIGS. 9B-9C).

### Example 9

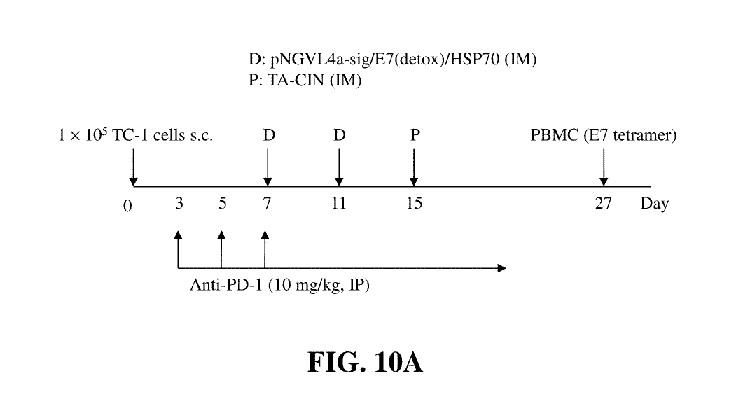

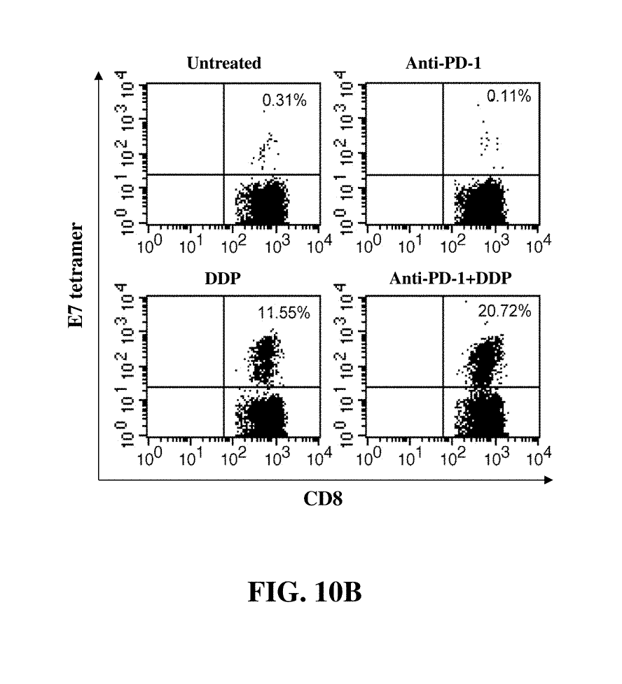

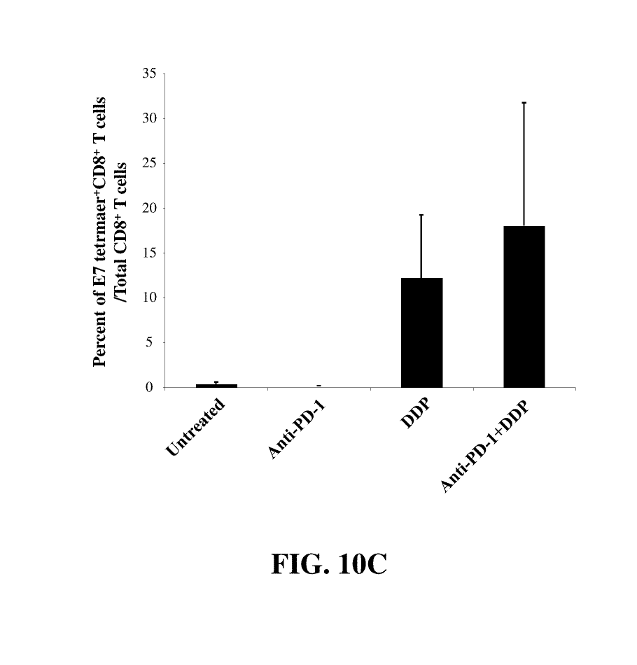

FIG. 10A illustrates a detection of the HPV16 E7-specific CD8+ T cells after the pNGVL4a-sig/E7(detox)/HSP70 DNA vaccine vaccination prime followed by the TA-CIN protein vaccination boost with or without the PD-1 blockade treatment in the HPV16 E6E7-expressing TC-1 tumor-bearing mice. The 5-8 weeks old female C57BL/6 mice (5 mice/group) are injected with 1×10⁵ TC-1 cells subcutaneously on day 0. On day 3, two groups of the tumor-bearing mice are injected with 10 mg/kg of the anti-PD-1 via intraperitoneally (IP) and repeated every two days. On day 7 and 11, the anti-PD-1 treated or untreated mice are vaccinated with 25 μg/mouse of the pNGVL4a-sig/E7(detox)/HSP70 DNA vaccine through intramuscular (IM) injection. These mice are further boosted with 25 μg/mouse of the TA-CIN protein through intramuscular injection on day 15. 12 days after the TA-CIN protein vaccination, PBMCs are prepared from the mice, and stained with purified anti-mouse CD16/32 antibody first. The PBMCs are then stained with FITC-conjugated anti-mouse CD8a antibody and PE-conjugated HPV16 E7aa49-57 peptide-loaded H-2D^{b} tetramer. The PBMCs are acquired with FACSCalibur and analyzed with CellQuest Pro software. FIG. 10B illustrates a representative flow cytometry analysis of the HPV16 E7-specific CD8+ T cells after the pNGVL4a-sig/E7(detox)/HSP70 DNA vaccine vaccination prime followed by the TA-CIN protein vaccination boost with or without the PD-1 blockade treatment in the HPV16 E6E7-expressing TC-1 tumor-bearing mice. FIG. 10C illustrates a quantitative summary of the flow cytometry data of the HPV16 E7-specific CD8+ T cells after the pNGVL4a-sig/E7(detox)/HSP70 DNA vaccine vaccination prime followed by the TA-CIN protein vaccination boost with or without the PD-1 blockade treatment in the HPV16 E6E7-expressing TC-1 tumor-bearing mice. The pNGVL4a-sig/E7(detox)/HSP70 DNA vaccine prime followed by the TA-CIN protein vaccination boost with the PD-1 blockade treatment is able to generate a robust response of the HPV type 16 E7-specific CD8+ T cell in the TC-1 tumor-bearing mice.

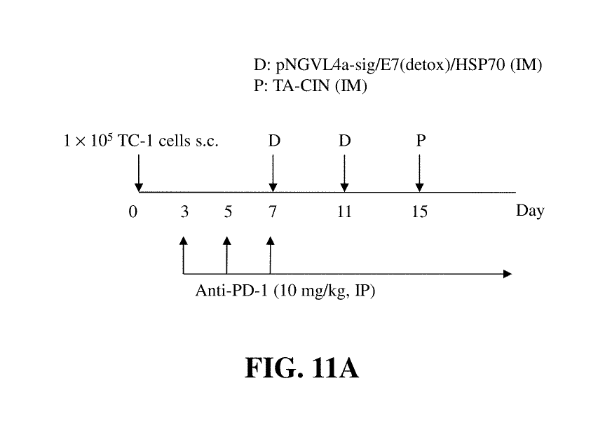

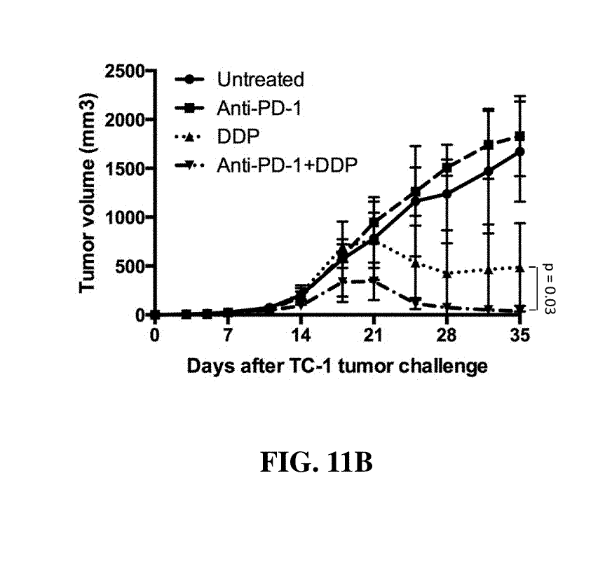

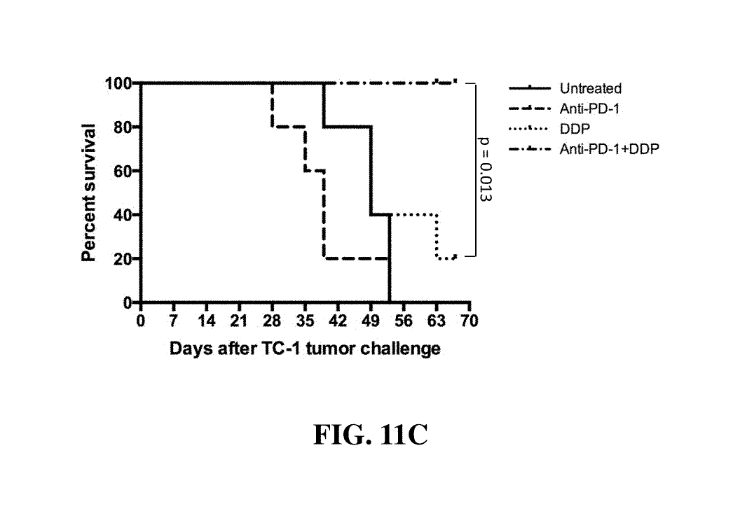

FIG. 11A illustrates anti-tumor effect and survival of the HPV16 E6E7-expressing TC-1 tumor-bearing mice after the pNGVL4a-Sig/E7(detox)/HSP70 DNA prime followed by the TA-CIN protein vaccination boost with or without the PD-1 blockade treatment. The 5-8 weeks old female C57BL/6 mice (5 mice/group) are injected with 1×10⁵ TC-1 cells subcutaneously on day 0. On day 3, two groups of the tumor-bearing mice are injected with 10 mg/kg of the anti-PD-1 via intraperitoneally (IP) and repeated every two days. On day 7 and 11, the anti-PD-1 treated or untreated mice are vaccinated with 25 μg/mouse of the pNGVL4a-sig/E7(detox)/HSP70 DNA vaccine through intramuscular (IM) injection. These mice are further boosted with 25 μg/mouse of the TA-CIN protein through intramuscular injection on day 15. The tumor growth is monitored twice a week with a digital caliper. FIG. 11B illustrates a quantitative summary of the TC-1 tumor volume in HPV type 16 E6/E7-expressing TC-1 tumor-bearing mice. The death of the mouse is recorded as either natural death or when the tumor size over 2 cm in diameter. FIG. 11C illustrates the duration of survival of HPV type 16 E6/E7-expressing TC-1 tumor-bearing mice receiving each treatment regimens. The mice treated with the pNGVL4a-sig/E7(detox)/HSP70 DNA prime followed by the TA-CIN protein vaccination boost with the anti-PD-1 treatment (anti-PD-1+DDP) show the lowest tumor volumes. Furthermore, the mice vaccinated with the anti-PD-1+DDP regimen show the longest overall survival. The results suggest that vaccination with the anti-PD-1+DDP regimen provides the most effective anti-tumor immunity and control of tumor growth in the HPV type 16 E6/E7-expressing TC-1 tumor-bearing mice (FIGS. 11B-11C).

The TA-CIN protein vaccination effectively elicits the HPV type 16-specific CD8+ T cell, the CD4+ T cell, and the antibody response (FIG. 4F). When the antigen-specific CD8+ T cell levels are very low, the anti-PD-1 is not able to have a significant effect, and the mice treated with the anti-PD-1 alone have similar tumor volume levels to the untreated mice (FIG. 5B). These data suggest that the TA-CIN vaccination enhanced the E7-specific CD8+ T cell response, which resultantly enabled the PD-1 blockade to further suppress tumor growth (FIG. 5B). Furthermore, the data suggest that vaccination with the TA-CIN protein in conjunction with the anti-PD-1 is the most effective at controlling tumor growth in the HPV type 16 E6E7-expressing TC-1 tumor-bearing mice (FIG. 5B). The individual successes of the combination comprising the TA-CIN protein and the anti-PD-1 demonstrated in this study shed light on the potential use of the TA-CIN protein and the PD-1 blockade to treat E6/E7-specific HPV-associated diseases.

This implies that the TA-CIN protein is not only able to induce a cell-mediated immune response, but it can also mediate a humoral immune response. Furthermore, the TA-CIN protein is used as both a prophylactic and therapeutic vaccine for the regression of HPV16+ tumors. Antigens presented in protein form are often directed towards the MHC class II pathway through exogenous antigen presentation. Since the TA-CIN protein is in filterable particulate form rather than soluble protein form, it is able to enter the MHC class II pathway through exogenous antigen presentation as well as the MHC class I pathway through cross presentation. The TA-CIN protein is capable of inducing a strong antigen-specific CD8+ T cell response without adjuvant. The results further suggest that the TA-CIN protein can induce a strong HPV type 16-specific CD8+ and CD4+ T cell response without adjuvant.

The anti-tumor activity of the anti-PD1 treatment in concert with the DNA vaccine vaccination is better than that of single either anti-PD1 treatment or the DNA vaccine vaccination.

The above examples suggest that immunotherapy with therapeutic vaccines targeting E6 and E7 combined with PD-1 blockade can increase the HPV type 16 E6/E7-specific CD8+ and CD4+ T cells. Furthermore, the present disclosure is the first to treat HPV type 16 E6E7-expressing TC-1 tumor-bearing mice using a combination comprising a TA-CIN protein vaccine or a DNA vaccine, and PD-1-related blockades.

While the present disclosure has been presented in accordance with several preferred and practical embodiments thereof, it is recognized that departures from the instant disclosure are fully contemplated within the spirit and scope of the invention.

The examples shown and described above are only examples. Many details are often found in the art. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, and including the full extent established by the broad general meaning of the terms used in the claims. It will therefore be appreciated that the examples described above may be modified within the scope of the claims.

1. An immunostimulatory combination comprising:
a PD-1-related blockade; and
a therapeutic vaccine, comprising a TA-CIN protein, a DNA vaccine, a recombinant viral vaccine, or any combination thereof in a heterologous prime-boost format,
wherein the immunostimulatory combination enhances a subject's immune responses against human papillomavirus (HPV) associated diseases.

2. (canceled)

3. The immunostimulatory combination of claim 1, wherein the TA-CIN protein is a single fusion protein comprising HPV proteins E6, E7, and L2.

4. The immunostimulatory combination of claim 1, wherein the DNA vaccine comprises a pNGVL4a-CRT/E6E7L2 DNA vaccine or a pNGVL4a-sig/E7(detox)/HSP70 DNA vaccine.

5. The immunostimulatory combination of claim 1, wherein the recombinant viral vaccine is a recombinant vaccinia viral vaccine expressing HPV16 and HPV18 E6 and E7 antigens.

6. The immunostimulatory combination of claim 1, wherein the PD-1-related blockade comprises a monoclonal anti-PD-1 or a monoclonal anti-PD-L1.

7. The immunostimulatory combination of claim 1, wherein the HPV comprises HPV types 6, 11, 16, 18, 31, 33, 35, 39, 45, 51, 52, 56, 58, 59, 66 or 68.

8. The immunostimulatory combination of claim 1, wherein the HPV-associated diseases comprise warts, papilloma, intraepithelial neoplasia, penile cancer, vaginal cancer, vulva cancer, anal cancer, oropharyngeal cancer or cervical cancer.

9. The immunostimulatory combination of claim 1, wherein the subject comprises human, mice, or other animal.

10. A method of administration to a subject in preferable amount of an immunostimulatory combination comprising a therapeutic vaccine and a PD-1-related blockade.

11. The method of claim 10, wherein the therapeutic vaccine comprises a TA-CIN protein, a DNA vaccine, or a recombinant viral vaccine, or any combination thereof in a heterologous prime-boost format.

12. The method of claim 11, wherein the TA-CIN protein is a single fusion protein consisting of HPV proteins E6, E7, and L2.

13. The method of claim 11, wherein a dosages ranging of the TA-CIN protein is from 1 microgram per milliliter to 500 microgram per milliliter.

14. The method of claim 11, wherein the DNA vaccine comprises a pNGVL4a-CRT/E6E7L2 DNA vaccine or a pNGVL4a-sig/E7(detox)/HSP70 DNA vaccine.

15. The method of claim 11, wherein a dosages ranging of the DNA vaccine is from 1 microgram per subject to 10 milligram per subject.

16. The immunostimulatory combination of claim 11, wherein the recombinant viral vaccine is a recombinant vaccinia viral vaccine expressing HPV16 and HPV18 E6 and E7 antigens.

17. The method of claim 11, wherein a dosages ranging of the recombinant viral vaccine is from 2×10⁴ pfu to 1×10⁷ pfu.

18. The method of claim 10, wherein the PD-1-related blockade comprises a monoclonal anti-PD-1 or a monoclonal anti-PD-L1.

19. The method of claim 10, wherein a dosages ranging of the PD-1-related blockade is from 1 milligram per kilogram to 100 milligram per kilogram.

20. The method of claim 10, wherein the HPV comprises HPV types 6, 11, 16, 18, 31, 33, 35, 39, 45, 51, 52, 56, 58, 59, 66 or 68.

21. The method of claim 10, wherein the HPV-associated diseases comprise warts, papillomas, intraepithelial neoplasia, penile cancer, vaginal cancer, vulva cancer, anal cancer, oropharyngeal cancer or cervical cancer.

22. The method of claim 10, wherein the subject comprises human, mice, or other animal.
